(19) United States
(12) Patent Application Publication
(10) Pub. No.: US 2026/0083901 A1
(43) Pub. Date: Mar. 26, 2026

(54) NEGATIVE PRESSURE WOUND THERAPY CANISTERS

(71) Applicant: Solventum Intellectual Properties Company, Maplewood, MN (US)

(72) Inventors: Benjamin A. PRATT, Poole (GB); Shannon C . INGRAM, San Antonio, TX (US); Joseph DARTNELL, Bracknell (GB)

(21) Appl. No.: 19/114,337

(22) PCT Filed: Sep. 19, 2023
(86) PCT No.: PCT/IB2023/059269

§ 371 Date: Mar. 21, 2025

Related U.S. Application Data
(60) Provisional application No. 63/413,872, filed on Oct. 6, 2022.

(51) Int. Cl. A61M 1/00

(52) U.S. Cl. CPC A61M 1/982; A61M 1/631; A61M 1/78; A61M 1/92

(57) ABSTRACT

A canister for use in a negative pressure wound therapy system. The canister includes a first fluid chamber, a second fluid chamber, and a filter disposed between the first fluid chamber and the second fluid chamber. The first fluid chamber is configured to receive fluids from a tissue site. The filter is configured to filter the fluids from the tissue site as the fluids move from the first fluid chamber to the second fluid chamber.

### CROSS-REFERENCE TO RELATED APPLICATIONS

This application is a U.S. National Stage Entry of International Application No. PCT/IB2023/059269, filed Sep. 19, 2023, which claims the benefit of priority to U.S. Provisional Application No. 63/413,872, filed on Oct. 6, 2022, each of which is incorporated herein by reference in its entirety.

### TECHNICAL FIELD

The invention set forth in the appended claims relates generally to tissue treatment systems and more particularly, but without limitation, to negative pressure wound therapy canisters.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for sensing changes at or near a wound site in a negative-pressure therapy environment are set forth in the appended claims. Illustrative embodiments are also provided to enable a person skilled in the art to make and use the claimed subject matter.

For example, in some embodiments, a canister for use in a negative pressure wound therapy system is described. The canister can include a first fluid chamber, a second fluid chamber, and a filter disposed between the first fluid chamber and the second fluid chamber. The first fluid chamber can be configured to receive fluids from a tissue site. The filter can be configured to filter the fluids from the tissue site as the fluids move from the first fluid chamber to the second fluid chamber.

In some example embodiments, the filter can include a filter carrier, a primary filter, and a secondary filter. The filter carrier can be configured to be coupled to the canister between the first fluid chamber and the second fluid chamber. The primary filter can be coupled to the first filter carrier and disposed proximate to the second fluid chamber. The secondary filter can be coupled to the filter carrier and disposed proximate to the first fluid chamber.

In some example embodiments, the canister can further include an instillation fluid pathway that can be configured to fluidly couple the second fluid chamber to the tissue site. In some example embodiments, the instillation fluid pathway can be disposed along an exterior of the canister. In some example embodiments, the instillation fluid pathway can be isolated from the first fluid chamber. In some example embodiments, the canister can further include a negative pressure pathway. The negative pressure pathway can be configured to fluidly couple the first fluid chamber to the tissues site. In some example embodiments, the negative pressure pathway can be isolated from the instillation fluid pathway. In some example embodiments, the canister can further include a fill pathway. The fill pathway can be configured to fluidly couple the second fluid chamber to an external fluid source. In some example embodiments, the fill pathway can be isolated from the negative pressure pathway and the instillation fluid pathway. In some example embodiments, the canister can further include a sensor disposed in the instillation fluid pathway. The sensor can be configured to generate a signal representative of a fill status of the second fluid chamber.

In some example embodiments, the canister can further include a first sterilization source and a second sterilization source. The first sterilization source can be configured to sterilize the first fluid chamber and the second sterilization source can be configured to sterilize the second fluid chamber. In some example embodiments, the first sterilization source and the second sterilization source are UV-C emitting devices.

In some example embodiments, the canister can further include a fluid altering device disposed within the second fluid chamber. The fluid altering device can be configured to release chemicals into fluids disposed within the second fluid chamber. In some example embodiments, the fluid altering device can be configured to sterilize the fluids within the second fluid chamber. In some example embodiments, the fluid altering device can be configured to chamber properties of the fluids within the second fluid chamber.

In some example embodiments, the canister can further include a negative pressure filter disposed within the first fluid chamber. The negative pressure filter can be configured to prevent liquids from the tissue site from contacting the negative pressure source.

In some example embodiments, the second fluid chamber can include a port. In some example embodiments, the port can be disposed at an end of the second fluid chamber opposite the first fluid chamber. In some example embodiments, the canister can further include a plug configured to removably couple to the port. In some example embodiments, the port can include a spout.

In some example embodiments, the canister can further include a sensor disposed in the first fluid chamber. The sensor can be configured to generate a signal representative of a fill status of the first fluid chamber.

Also described herein is a system for treating a tissue site. The system can include a dressing, a negative-pressure source, and a canister. The dressing can be configured to be disposed at the tissue site. The negative-pressure source can be configured to be fluidly coupled to the dressing and further configured to generate a negative pressure at the tissue site. The canister can be configured to be fluidly coupled between the dressing and the negative-pressure source. The canister can include a first fluid chamber, a second fluid chamber, and a filter. The first fluid chamber can be configured to receive fluids from the tissue site and the second fluid chamber can be configured to store fluids. The filter can be disposed between the first fluid chamber and the second fluid chamber. The filter can be configured to filter the fluids from the tissue site as the fluids move through the filter from the first fluid chamber to the second fluid chamber.

In some embodiments, the system can further include an instillation fluid pathway configured to fluidly couple the second fluid chamber to the tissue site. In some example embodiments, the system can further include a negative pressure pathway configured to fluidly couple the negative-pressure source to the dressing and the first fluid chamber of the canister. The negative pressure pathway can be isolated from the instillation fluid pathway.

Also described herein is a method of treating a tissue site. The method can include disposing a dressing at a tissue site, fluidly coupling a negative-pressure source to the dressing, and fluidly coupling a canister between the negative-pressure source and the dressing. The canister can include a first fluid chamber, a second fluid chamber, and a filter. The first fluid chamber can be configured to collect fluids from the tissue site. The filter can be disposed between the first fluid chamber and the second fluid chamber. The filter can be configured to filter the fluids from the tissue site as the fluids move through the filter from the first fluid chamber to the second fluid chamber. The method can further include operating the negative-pressure source to generate a negative pressure at the dressing, drawing fluids from the tissue site into the first fluid chamber of the canister in response to the negative pressure, and filtering, with the filter, the fluids from the tissue site as the fluids move from the first fluid chamber to the second fluid chamber.

In some example embodiments, the method can further include instilling the filtered fluids of the second fluid chamber to the tissue site.

In some example embodiments, the method can further include disposing of the filtered fluids of the second fluid chamber. In some example embodiments, disposing of the filtered fluids of the second fluid chamber can include removing a plug from an emptying port of the second fluid chamber and drawing the filtered fluids from the second fluid chamber through the emptying port.

In some example embodiments, the method can further include purifying the fluids in the first fluid chamber with a UV-C source.

In some example embodiments, the method can further include treating the filtered fluids of the second fluid chamber with a UV-C source.

In some example embodiments, the second fluid chamber can be configured to receive and house instillation fluid.

In some example embodiments, the method can further include filling the second fluid chamber with instillation fluid. In some example embodiments, the method can further include fluidly coupling the second fluid chamber to the dressing and instilling the instillation fluid from the second fluid chamber to the dressing. In some example embodiments, filling the second fluid chamber with the instillation fluid can include adding the instillation fluid from an external fluid source through a port of the second fluid chamber. In some example embodiments, filling the second fluid chamber with instillation fluid includes coupling a fluid pathway of the canister to an external fluid source and operating a pump to draw fluid from the external fluid source to the second fluid chamber.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2A is an exploded view of a recirculatory canister that may be associated with some embodiments of FIG. 1, illustrating additional details that may be associated with some example embodiments;

FIG. 2B is a front view of the recirculatory canister of FIG. 2A, illustrating additional details that may be associated with some example embodiments;

FIG. 2C is a side view of the recirculatory canister of FIG. 2A, illustrating additional details that may be associated with some example embodiments;

FIG. 2D is a back view of the recirculatory canister of FIG. 2A, illustrating additional details that may be associated with some example embodiments;

FIG. 2E is a cross-sectional view of the recirculatory canister of FIG. 2A taken along line 2E-2E of FIG. 2D, illustrating additional details that may be associated with some example embodiments;

FIG. 3A is a cross-sectional view of the recirculatory canister of FIG. 2A taken along line 2E-2E of FIG. 2D, illustrating an instillation mode of operation;

FIG. 3B is a cross-sectional view of the recirculatory canister of FIG. 2A taken along line 2E-2E of FIG. 2D, illustrating a negative-pressure mode of operation;

FIG. 3C is a cross-sectional view of the recirculatory canister of FIG. 2A taken along line 2E-2E of FIG. 2D, illustrating fluids filtering from a first fluid chamber of the canister to a second fluid chamber of the canister;

FIG. 3D is a cross-sectional view of the recirculatory canister of FIG. 2A taken along line 2E-2E of FIG. 2D, illustrating the second fluid chamber of the canister housing fluids;

FIG. 4A is an exploded view of a reusable canister that may be associated with some embodiments of FIG. 1, illustrating additional details that may be associated with some example embodiments;

FIG. 4B is a front view of the reusable canister of FIG. 4A, illustrating additional details that may be associated with some example embodiments;

FIG. 4C is a back view of the reusable canister of FIG. 4A, illustrating additional details that may be associated with some example embodiments;

FIG. 4D is a side view of the reusable canister of FIG. 4A, illustrating additional details that may be associated with some example embodiments;

FIG. 4E is a cross-sectional view of the reusable canister of FIG. 4A taken along line 4E-4E of FIG. 4C, illustrating additional details that may be associated with some example embodiments;

FIG. 4F is a cutaway view illustrating a second fluid chamber of the reusable canister of FIG. 4A, illustrating additional details that may be associated with some example embodiments;

FIG. 5A is a cross-sectional view of the reusable canister of FIG. 4A taken along line 4E-4E of FIG. 4C, illustrating a negative-pressure mode of operation;

FIG. 5B is a cross-sectional view of the reusable canister of FIG. 4A taken along line 4E-4E of FIG. 4C, illustrating fluids filtering from a first fluid chamber of the reusable canister to a second fluid chamber of the reusable canister;

FIG. 5C is a cross-sectional view of the reusable canister of FIG. 4A taken along line 4E-4E of FIG. 4C, illustrating fluids being removed from the second fluid chamber of the reusable canister;

FIG. 6A is a perspective view of another embodiment of a reusable canister of FIG. 1, illustrating additional details that may be associated with some example embodiments;

FIG. 6B is an exploded view of the reusable canister of FIG. 6A, illustrating additional details that may be associated with some example embodiments;

FIG. 6C is a perspective view of another embodiment of a reusable canister of FIG. 1, illustrating additional details that may be associated with some example embodiments;

FIG. 6D is an exploded view of the reusable canister of FIG. 6C, illustrating additional details that may be associated with some example embodiments;

FIG. 6E is a perspective view of another embodiment of a reusable canister of FIG. 1, illustrating additional details that may be associated with some example embodiments;

FIG. 6F is an exploded view of the reusable canister of FIG. 6E, illustrating additional details that may be associated with some example embodiments;

FIG. 6G is a perspective view of another embodiment of a reusable canister of FIG. 1, illustrating additional details that may be associated with some example embodiments;

FIG. 6H is an exploded view of the reusable canister of FIG. 6G, illustrating additional details that may be associated with some example embodiments;

FIG. 7 is a side cross-sectional view of another embodiment of a recirculatory canister of FIG. 1, illustrating additional details that may be associated with some example embodiments; and

FIG. 8 is a perspective view of an embodiment of a recirculatory and reusable canister that may be associated with the therapy system of FIG. 1, illustrating additional details that may be associated with some example embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

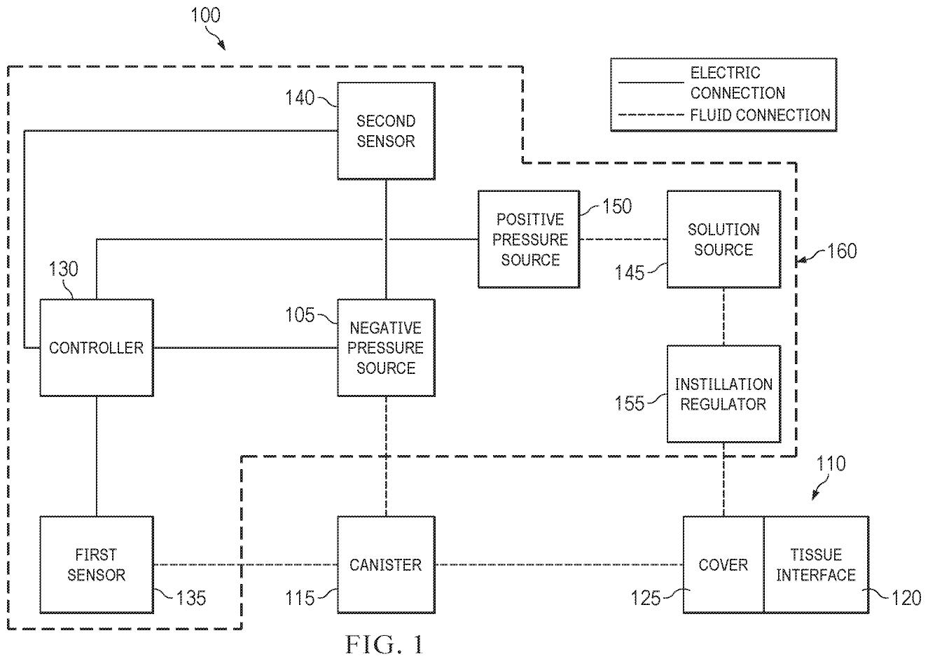

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a canister 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT. R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g., saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit 160.

In general, components of the therapy system 100 may be coupled directly or indirectly. For example, the negative-pressure source 105 may be directly coupled to the canister 115 and may be indirectly coupled to the dressing 110 through the canister 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The canister 115 is representative of a container, canister, pouch, or other storage component, which can be used to manage exudates and other fluids withdrawn from a tissue site. In many environments, a rigid canister may be preferred or required for collecting, storing, and disposing of fluids. In other environments, fluids may be properly disposed of without rigid canister storage, and a re-usable canister could reduce waste and costs associated with negative-pressure therapy.

A controller, such as the controller 130, may be a microprocessor or a computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be an apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source.

In some example embodiments, the cover 125 may be a polymer drape, such as a polyurethane film, that is permeable to water vapor but impermeable to liquid. In other embodiments, the cover 125 may be impermeable to both water vapor and liquids. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S. A., Colombes, France; and INSPIRE® 2301 and INSPIRE® 2327 polyurethane films, commercially available from Exopack Advanced Coatings, Wrexham, United Kingdom. In some embodiments, the cover 125 may comprise INSPIRE® 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in the canister 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. In some example embodiments, the controller 130 can operate the negative-pressure source 105 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of +25 mmHg/min. and a descent rate set at −25 mmHg/min. In other embodiments of the therapy system 100, the triangular waveform may vary between negative pressure of 25 and 135 mmHg with a rise rate of about +30 mmHg/min and a descent rate or about −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second and 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 150 to move solution from the solution source 145 to the tissue interface 120. Additionally or alternatively, the solution source 145 may be elevated to a height sufficient to allow gravity to move solution into the tissue interface 120.

The controller 130 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 120, or it may be implemented to provide a dynamic pressure mode of operation to vary the flow rate of instillation solution through the tissue interface 120. Alternatively, the application of negative pressure may be implemented to provide an intermittent mode of operation to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied. The controller 130 may be utilized to select a mode of operation and the duration of the negative pressure treatment before commencing another instillation cycle by instilling more solution.

Negative-pressure therapy and instillation therapy may increasingly be performed in home environments across more geographies. Many current canisters and instillation fluid sources for therapy systems are one-time use devices intended for us in a hospital or clinic setting. As the devices near their usable life, the canisters and instillation fluid sources may be removed form a therapy system and replaced. The removal and replacement of canisters and instillation fluid sources from a therapy system may involve complex fluid connections that may be most appropriately addressed by a trained clinician. In a home environment, it may be difficult for a patient to handle the canister and instillation fluid source removal and replacement that are required to fully treat the tissue site. Some systems require interaction with many different devices which may be cumbersome. In a home environment, some patients may be concerned about the environmental impact of the disposal of these devices during treatment of the tissue site. The canister 115 may address these and other issues by providing a reusable and/or recirculatory system that can house both wound exudate and instillation fluid. In some embodiments, the canister 115 may be capable of filtering and/or purifying exudate to produce a fluid suitable for disposal down a drain or reuse of the purified fluid to cleanse the tissue site.

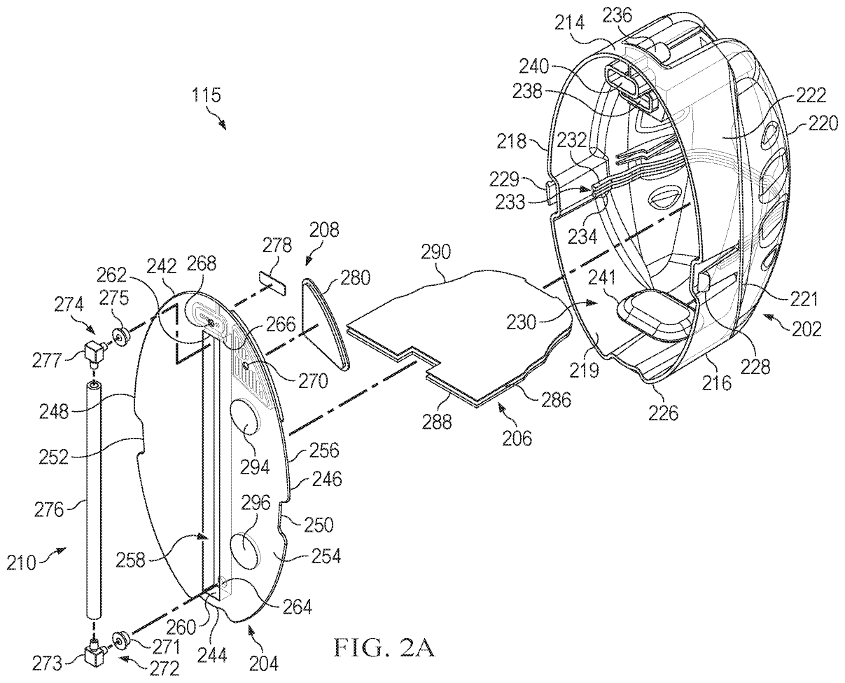

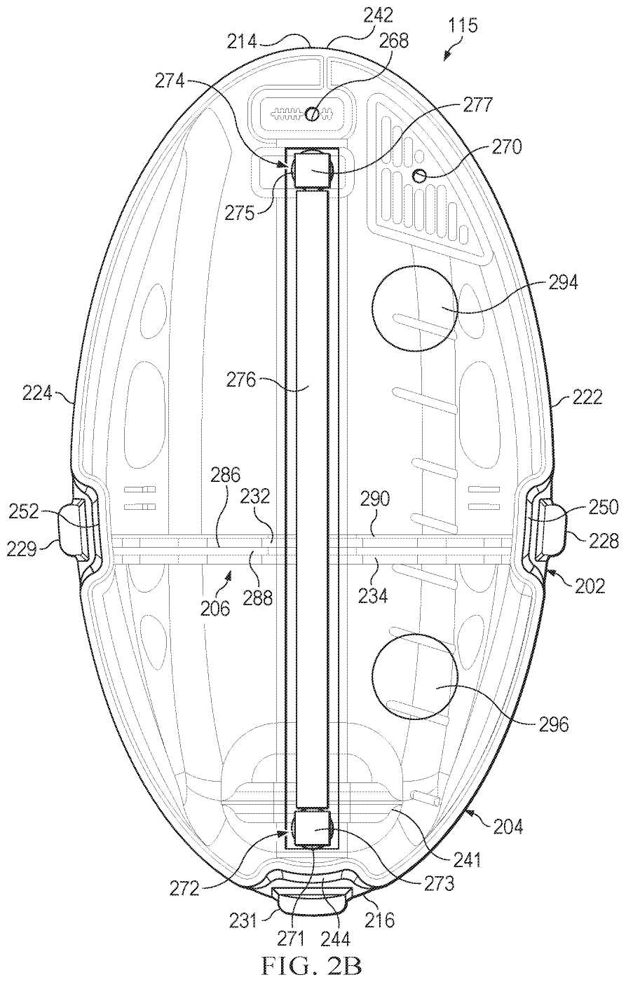

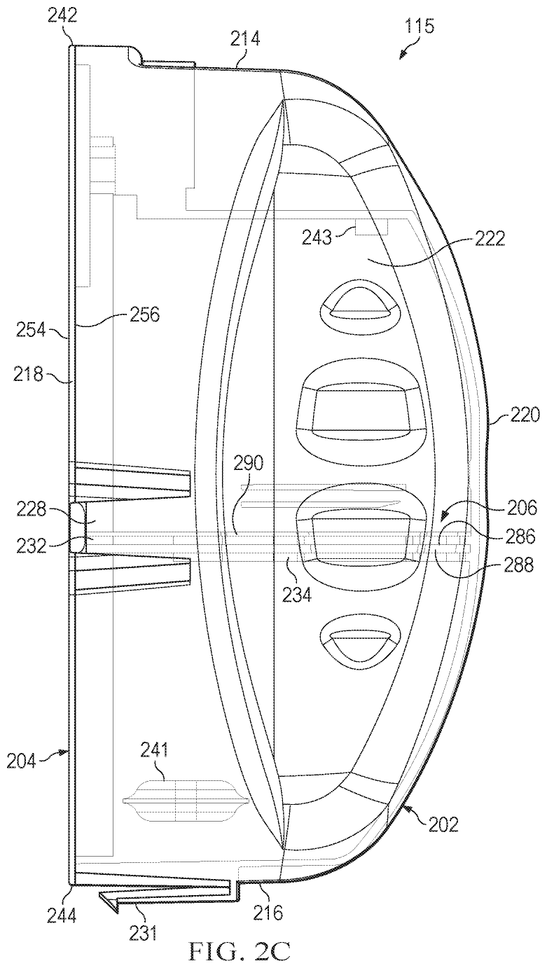

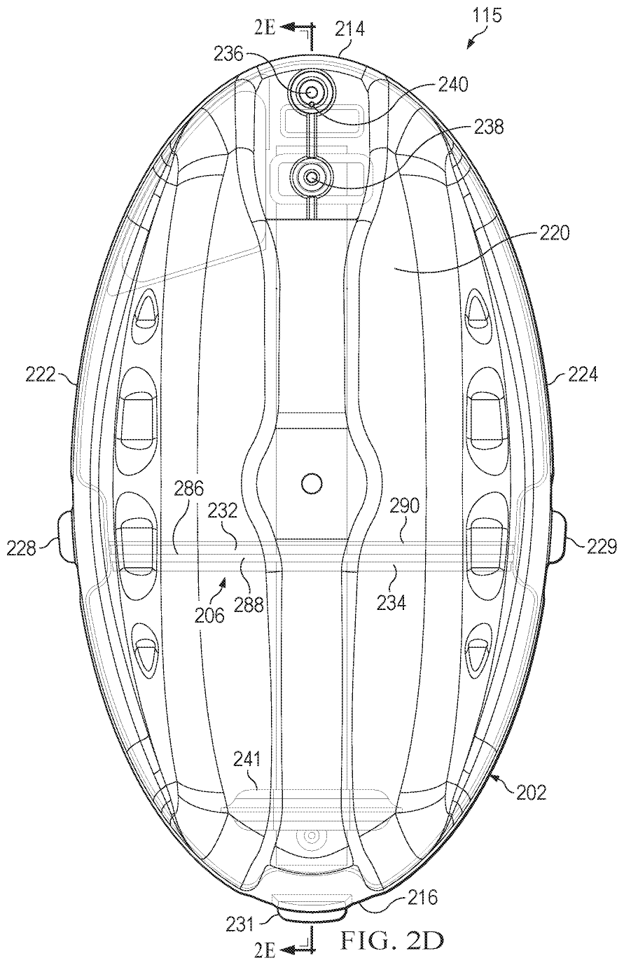

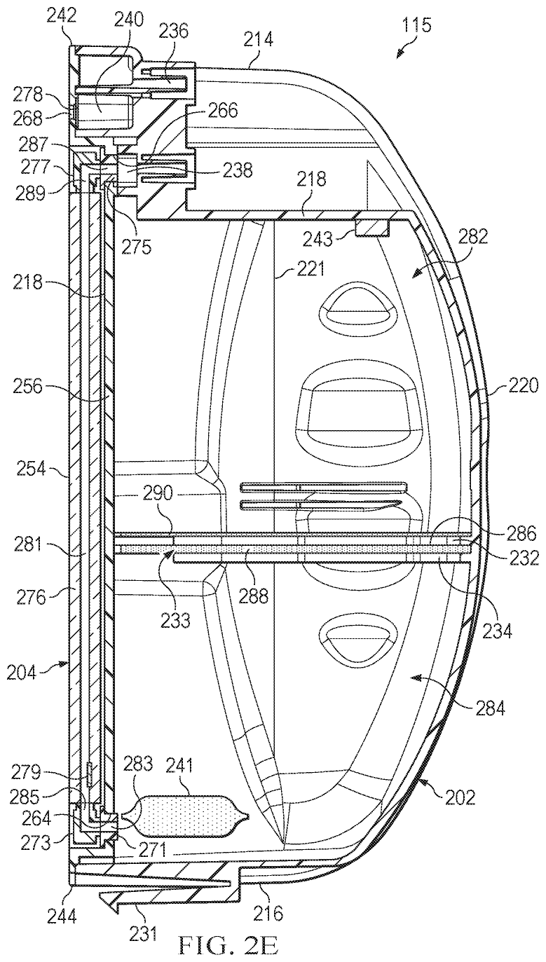

FIGS. 2A-2E illustrate various views of an exemplary embodiment of the canister 115 of the therapy system 100 of FIG. 1. In some embodiments, the canister 115 may be a recirculatory canister. FIG. 2B is a front view of the canister 115. FIG. 2C is a side view of the canister 115. FIG. 2D is a back view of the canister 115. FIG. 2E is a cross-sectional view of the canister 115 taken along line 2E-2E of FIG. 2D.

FIG. 2A is an exploded view of the canister 115 illustrating additional details that may be associated with some embodiments. The canister 115 may include a canister body 202, a canister plate 204, a filter 206, one or more negative pressure filters 208, and an instillation system 210. The canister body 202 may at least partially form an interior 230. In some embodiments, the canister body 202 may have a stadium or ovular shape. In other embodiments, the canister body 202 may be other shapes having the interior 230. The interior 230 may be configured to receive and retain fluids within the canister 115 after assembly of the canister 115.

In some embodiments, the canister body 202 may include a first wall 218 and a second wall 220. The second wall 220 may be semi-ovoid in shape. The first wall 218 may be an annular wall having an ovular shape and have a first end coupled to an edge 221 of the second wall 220 formed by a plane creating the semi-ovoid shape of the second wall 220. In some embodiments, a second end of the first wall 218 may form an opening 219 into the interior 230 of the canister body 202. The second end of the first wall 218 may be configured to receive the canister plate 204. The canister body 202 may include a first end 214 and a second end 216 opposite the first end 214. The first end 214 may be a top end or surface of the canister body 202, and the second end 216 may be a bottom end or surface of the canister body 202. The canister body 202 may have a first side 222 and a second side 224 extending between the first end 214 and the second end 216. The first side 222 and the second side 224 may be opposite one another and may form portions of an exterior surface of the canister body 202. In some embodiments, the first side 222 and the second side 224 may generally be symmetrical to each other. The first side 222 and the second side 224 may have variation in symmetry to accommodate of other elements of the canister 115

In some embodiments, the first wall 218 of the canister body 202 may include a coupling edge 226. For example, the coupling edge 226 may be the second end of the first wall 218 opposite the second wall 220. The coupling edge 226 may be configured to be coupled to the canister plate 204 to close the interior 230.

The canister body 202 may also include one or more connectors such as a first connector 228, a second connector 229, and a third connector 231. In some embodiments, the first connector 228 may be disposed on the first side 222 of the canister body 202 and the second connector 229 may be disposed on the second side 224 of the canister body 202. The first connector 228 and the second connector 229 may be coupled to the first wall 218 of the canister body 202. In some embodiments, the first connector 228 and the second connector 229 may be aligned with each other between the first end 214 and the second end 216. For example, the first connector 228 may be positioned approximately halfway between the first end 214 and the second end 216. Similarly, the second connector 229 may be positioned approximately halfway between the first end 214 and the second end 216. In some embodiments, the first connector 228 and the second connector 229 may be disposed in recesses formed in an exterior surface of the first wall 218. The third connector 231 may be coupled to the second end 216 of the canister body 202. The third connector may be positioned centrally on the second end 216 between the first side 222 and the second side 224. The first connector 228 and, the second connector 229, and the third connector 231 may comprise releasable latches permitting the canister 115 to be coupled to and to be separated from the therapy system 100. In some embodiments, the first connector 228 and, the second connector 229, and the third connector 231 may be a portion of a cantilever snap-fit type latch that is configured to be inserted into a receiver of the therapy system 100.

The canister body 202 may include features within an interior 230 of the canister body 202 to provide structure for elements such as the filter 206. For example, the canister body 202 may include a channel 233 formed by a first shelf 232 and a second shelf 234. The first shelf 232 and the second shelf 234 may be coupled to the first wall 218 and the second wall 220 on a surface of the first wall 218 and the second wall 220 facing the interior 230. In some embodiments, the first shelf 232 and the second shelf 234 extend from the first side 222 of the canister body 202 to the second side 224 of the canister body 202. The first shelf 232 and the second shelf 234 may be generally parallel to each other so that a width of the channel 233 is substantially constant. The filter 206 may be disposed within the channel 233 and may divide the canister 115 into a first fluid chamber 282 and a second fluid chamber 284. The first fluid chamber 282 may be configured to receive fluids from a tissue site and may be disposed between the filter 206 and the first end 214 of the canister body 202. The second fluid chamber 284 may be disposed between the filter 206 and the second end 216 of the canister body 202.

The canister body 202 may additionally include at least one communication element. In some embodiments, the at least one communication element may be a fluid inlet 236 disposed at or proximate to the first end 214 of the canister body 202. The fluid inlet 236 may be a port having at least one channel or lumen within the fluid inlet 236 to permit fluids to flow across one or more of the first wall 218 and the second wall 220 between the exterior environment and the interior 230. In some embodiments, the fluid inlet 236 may be disposed in a recess formed in an exterior of the first wall 218 proximate to the first end 214. The at least one communication element may also include a fluid outlet 238. The fluid outlet 238 may be a port having at least one channel or lumen within the fluid outlet 238 to permit to flow across one or more of the first wall 218 and the second wall 220 from the interior 230 of the canister body 202 to an exterior environment. In some embodiments, the canister body 202 may additionally include a sensing line 240. The sensing line 240 may comprise a channel, lumen, or other fluid pathway from the therapy unit 160, through the canister 115 to permit the therapy unit 160 to sense a pressure at the dressing 110.

In some embodiments, the canister body 202 may optionally include a fluid altering device 241. The fluid altering device 241 may be disposed within the second fluid chamber 284. The fluid altering device 241 may be configured to change properties of the fluids within the second fluid chamber 284. For example, in some embodiments, the fluid altering device 241 may be configured to sterilize the fluids within the second fluid chamber 284. Additionally or alternatively, the fluid altering device 241 may be configured to create saline out of fluid stored in the second fluid chamber 284. In some embodiments, the fluid altering device 241 may be configured to alter the properties of the fluids based on what would be beneficial for the tissue site being treated by the therapy system 100.

In some embodiments, the canister body 202 may optionally include a first sensor 243. The first sensor 243 may be disposed in the first fluid chamber 282. In some embodiments, the first sensor 243 may be configured to configured to generate a signal indicative of a fluid level in the first fluid chamber 282. In some embodiments, the first sensor 243 may be communicatively coupled to at least the controller 130 of the therapy system 100 such that the controller 130 may receive the signal indicative of the fluid level in the first fluid chamber 282 and operate other components of the therapy system 100 in response.

The canister plate 204 may be stadium or ovular in shape or may be another shape that aligns with the coupling edge 226 of the first wall 218 of the canister body 202. The canister plate 204 may have a first end 242, a second end 244 opposite the first end 242. The canister plate 204 may include a first side 246 extending from the first end 242 to the second end 244, and a second side 248 opposite the first side 246. The canister plate 204 may have a first indentation 250 in the first side 246 and a second indentation 252 in the second side 248. Both the first indentation 250 and the second indentation 252 may align with the recesses in the first wall 218 in which the first connector 228 and the second connector 229 of the canister body 202 are disposed.

The canister plate 204 may additionally include an exterior surface 254 and an interior surface 256 opposite the exterior surface 254. The exterior surface 254 of the canister plate 204 may include a channel 258 that may be configured to receive one or more components of the instillation system 210. The channel 258 may have a first end 260 and a second end 262 opposite the first end 260. A first end 260 may include a first opening 264 and the second end 262 may include a second opening 266. In some embodiments, the canister plate 204 may be coupled to the coupling edge 226 of the first wall 218, and the second opening 266 may be aligned with the fluid outlet 238 of the canister body 202.

The canister plate 204 may also include a pressure sensor opening 268 and a negative pressure opening 270. The pressure sensor opening 268 and the negative pressure opening 270 may each be located proximate to the first end 242 of the canister plate 204. In some embodiments, the pressure sensor opening 268 may be located centrally between the first side 246 and the second side 248 and between the channel 258 and the first end 242 of the canister plate 204. The negative pressure opening 270 may be located between the pressure sensor opening 268 and the first side 246. In some embodiments, the canister plate 204 may be coupled to the coupling edge 226 of the first wall 218, and the pressure sensor opening 268 may be fluidly coupled to the sensing line 240, and the negative pressure opening 270 may be fluidly coupled to the interior 230 of the canister body 202.

In some embodiments, the canister plate 204 may optionally include one or more sterilizations sources, such as a first sterilization source 294 and a second sterilization source 296. In some embodiments, a first sterilization source 294 may be located proximate to the first fluid chamber 282 and the second sterilization source 296 may be located proximate to the second fluid chamber 284. The one or more sterilization sources may be configured to sterilize the first fluid chamber 282 and the second fluid chamber 284. For example, the one or more sterilization sources may be configured to sterilize, reduce, or eliminate any bacteria, mold, viruses, or other potentially harmful contaminants located within the canister 115. In some embodiments, the one or more sterilization sources may be diodes such as UV-C emitting diodes. In some embodiments, the UV-C emitting diodes may emit electromagnetic radiation in a wavelength range of between about 100 nanometers and about 280 nanometers.

In some embodiments, the exterior surface 254 of the canister plate 204 may be configured to couple with the therapy unit 160. In some embodiments, the first sterilization source 294 and the second sterilization source 296 may be windows through the canister plate 204. The therapy unit 160 may include one or more sterilization sources such as UV-C emitting diodes that may be configured to align with the windows through the canister plate 204. The windows may be configured to transmit electromagnetic radiation from the one or more sterilization sources emitting diodes to the interior 230 of the canister body 202. In some embodiments, there may be one sterilization source that may be configured to transmit electromagnetic radiation through each of the windows in the canister plate 204. Alternatively, there may be more than one sterilization source.

The one or more sterilization sources may be communicatively coupled with the controller 130 of the therapy unit 160. The one or more sterilization sources may be configured to be actuated to sterilize the interior 230 of the canister body 202. In some embodiments, the first sterilization source 294 may be configured to sterilize the first fluid chamber 282 and the second sterilization source 296 may be configured to sterilize the second fluid chamber 284.

The instillation system 210 may include an inlet 272, an outlet 274, and a conduit 276. The inlet 272 may couple the conduit 276 to the first opening 264. In some embodiments, the inlet 272 may provide a fluid path from a lumen 281 of the conduit 276 to the first opening 264. The inlet 272 may include a first piece 271 and a second piece 273. The first piece 271 may be a grommet or other seal configured to couple the inlet 272 to the first opening 264 while maintaining a fluid seal between a fluid passage through the inlet 272 and the first opening 264. The second piece 273 may be an elbow connector or other device configured to receive fluid flowing in a first direction and direct the fluid into a second direction. There may be a first lumen 283 extending through the first piece 271 and a second lumen 285 extending through the second piece 273. The first lumen 283 and the second lumen 285 may provide a path for fluid to move through the first piece 271 and the second piece 273.

The outlet 274 may couple the conduit 276 to the second opening 266. In some embodiments, the outlet 274 may provide a fluid path from a lumen of the conduit 276 to the second opening 266. The outlet 274 may include a first piece 275 and a second piece 277. The first piece 275 may be a grommet or other seal configured to couple the outlet 274 to the second opening 266 while maintaining a fluid seal between a fluid passage through the outlet 274 and the second opening 266. The second piece 277 may be an elbow connector or other device configured to receive fluid flowing in a first direction and direct the fluid into a second direction. There may be a first lumen 287 extending through the first piece 275 and a second lumen 289 extending through the second piece 277. The first lumen 287 and the second lumen 289 may provide a path for fluid to move through the first piece 275 and the second piece 277.

In some embodiments, the instillation system 210 may further include a second sensor 279. The second sensor 279 may be configured to generate a signal representative of a fill status of the second fluid chamber 284. For example, the second sensor 279 may be configured to sense when the second fluid chamber 284 is empty. In some embodiments, the second sensor 279 may be communicatively coupled to at least the controller 130 of the therapy system 100 such that the controller 130 may stop instillation therapy of the therapy system 100 if the controller 130 receives a signal from the second sensor 279 that indicates that the second fluid chamber 284 is empty.

In some embodiments, the instillation system 210 may additionally include one or more valves. The valves may be one-way valves and may be positioned within the instillation system 210 to prevent fluid from flowing into the canister 115 through the instillation system 210. In some embodiments, the components of the instillation system 210 may be formed from plastics, polymers, thermoplastics, metals, metal alloys, composition material, fiber-type materials, and other similar materials.

In some embodiments, the one or more negative pressure filters 208 may include a first filter 278 and a second filter 280. The first filter 278 may be configured to be positioned adjacent to the interior surface 256 of the canister plate 204 to cover the pressure sensor opening 268. The second filter 280 may be configured to be positioned adjacent to the interior surface 256 of the canister plate 204 to cover the negative pressure opening 270. The first filter 278 may be a liquid-air separator and be configured to prevent liquid and exudate from the interior 230 of the canister body 202 from exiting the canister 115 through the pressure sensor opening 268. The second filter 280 may be a liquid-air separator and be configured to prevent liquid and exudate from the interior 230 of the canister body 202 from exiting the canister 115 through the negative pressure opening 270.

In some embodiments, the filter 206 may be positioned in the channel 233 and retained in position between the first shelf 232 and the second shelf 234. In some embodiments, the filter 206 may be supported by the first shelf 232 and the second shelf 234 of the canister body 202. The filter 206 may include a filter carrier 286, a primary filter 288, and a secondary filter 290. The filter carrier 286 may be disposed between the primary filter 288 and the secondary filter 290 and may be configured to provide support to one or both of the primary filter 288 and the secondary filter 290. The primary filter 288 may be coupled to the filter carrier 286 and disposed proximate to the second fluid chamber 284. The secondary filter 290 may be proximate to or coupled to the filter carrier 286 and may be disposed proximate to the first fluid chamber 282. In some embodiments, the secondary filter 290 may be configured to rest on the first shelf 232 and the filter carrier 286 and the primary filter 288 may be disposed between the first shelf 232 and the second shelf 234.

The filter 206 may be configured to filter fluids from the tissue site as the fluids move from the first fluid chamber 282 to the second fluid chamber 284. In some embodiments, the filter 206 may include materials that are capable of physically filtering fluid such that water (H₂O) molecules may pass through the filter 206 while larger bacterial molecules are captured by the filter 206. Additionally, one or more of the filter carrier 286, the primary filter 288, or the secondary filter 290 of the filter 206 may be positively or negatively charged to capture bacteria while allowing plasma to pass through the filter 206.

The instillation system 210 may be configured to fluidly couple the second fluid chamber 284 to the dressing 110. More specifically, fluids in the second fluid chamber 284 may be transported from the second fluid chamber 284, through the instillation system 210, through the fluid outlet 238, and to the dressing 110 at the tissue site. The instillation system 210 may be configured to transport fluids of the second fluid chamber 284 to the dressing 110 while remaining fluidly isolated from the first fluid chamber 282.

In some embodiments, the canister 115 may be manufactured such that it is received by a user or a health care practitioner with instillation fluid within the second fluid chamber 284. Additionally or alternatively, the canister 115 may be received by a user or a health care practitioner without any fluid in the second fluid chamber 284. Instillation fluid may be introduced into the second fluid chamber 284 prior to using the canister 115 to treat a tissue site. In some embodiments, prior to using the canister 115 and the therapy system 100 to treat the tissues site, the canister 115 may be coupled to an external fluid source. More specifically, the external fluid source may be coupled by a tube, a conduit, or another element to the instillation system 210. The controller 130 may be configured to operate the positive-pressure source 150 in reverse such that instillation fluid stored in the external fluid source may be pulled from the external fluid source into the second fluid chamber 284 of the canister 115. For example, fluid may flow from the external fluid source through the tube or conduit to reach the fluid outlet 238. From the fluid outlet 238, the fluid may flow through the first piece 275 and the second piece 277 of the outlet 274, through the conduit 276, and through the second piece 273 and the first piece 271 of the inlet 272 to reach the second fluid chamber 284. Once the instillation fluid is stored in the second fluid chamber 284, the therapy system 100 may be capable of instilling the instillation fluid from the second fluid chamber 284 to the dressing 110.

In some embodiments, instillation fluid may not be introduced into the canister 115 prior to operating the therapy system 100 to treat a tissue site. The therapy system 100 may operate to draw the dressing 110 to a desired negative pressure which may draw fluid from the dressing 110 into the first fluid chamber 282 of the canister 115. The fluid may be filtered through the filter 206 as described above such that purified or filtered fluid may be stored in the second fluid chamber 284. Once a predetermined amount of fluid is stored within the second fluid chamber 284, the therapy system 100 may be capable of instilling the fluid from the second fluid chamber 284 to the dressing 110.

In some embodiments, the canister 115 may further include a communication element that may be communicatively coupled with the therapy unit 160 of the therapy system 100. In some embodiments, the communication element may utilize RFID technology which may enable the therapy system 100 to only be used with a particular patient and/or therapy unit. For example, the communication element may be configured to track one or more devices and/or patients the canister 115 has been used with and keep a record of the patients and lifespan of the canister 115. This information may be configured to be stored in a database that may be accessible by health care providers. The communication element of the canister 115 may also track the number of dressings that the canister 115 is coupled to. In some embodiments, the data may be used to optimize performance of the canister 115.

In some embodiments, the canister body 202 and the canister plate 204 may be transparent. In other embodiments not pictured herein, portions of the canister body 202 and/or the canister plate 204 may be transparent and portions may be opaque or the canister body 202 and the canister plate 204 can be opaque. The canister body 202 and the canister plate 204 may be formed from plastics, polymers, thermoplastics, metals, metal alloys, composition material, fiber-type materials, and other similar materials. The plastics described herein may be a substance or structure capable of being shaped or molded with or without the application of heat, a high polymer, usually synthetic, combined with other ingredients such as curatives, fillers, reinforcing agents, plasticizers, etc. Plastics can be formed or molded under heat and pressure in its raw state and machined to high dimensional accuracy, trimmed and finished in its hardened state. The thermoplastic type can be resoftened to its original condition by heat. In addition, the plastics may mean engineered plastics such as those that are capable of sustaining high levels of stress and are machinable and dimensionally stable. Some exemplary plastics are nylon, acetyls, polycarbonates, ABS resins, PPO/styrene, ISOPLAST 2530, TURLUX HS 2822, and polybutylene terephthalate. The thermoplastics described herein may be high polymers that soften when exposed to heat and return to their original condition when cooled to room temperature.

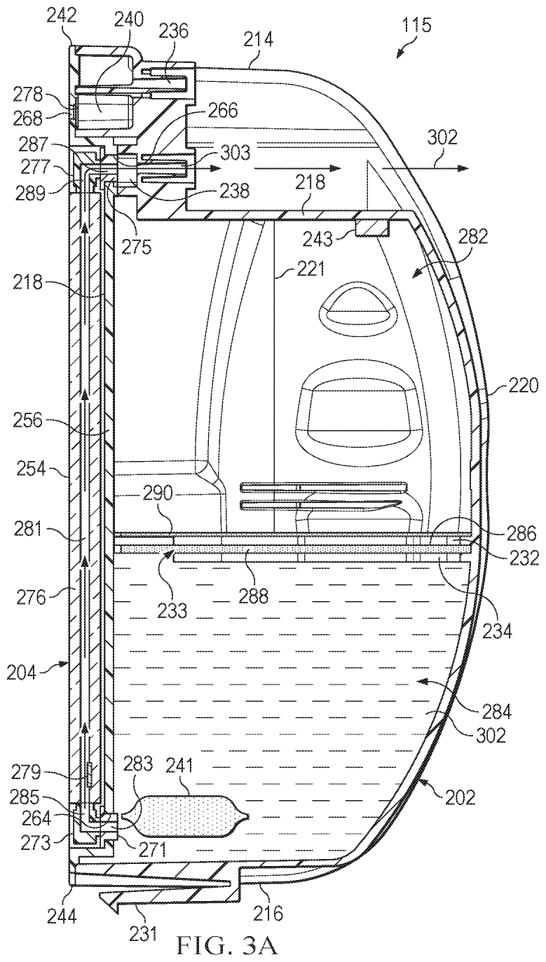

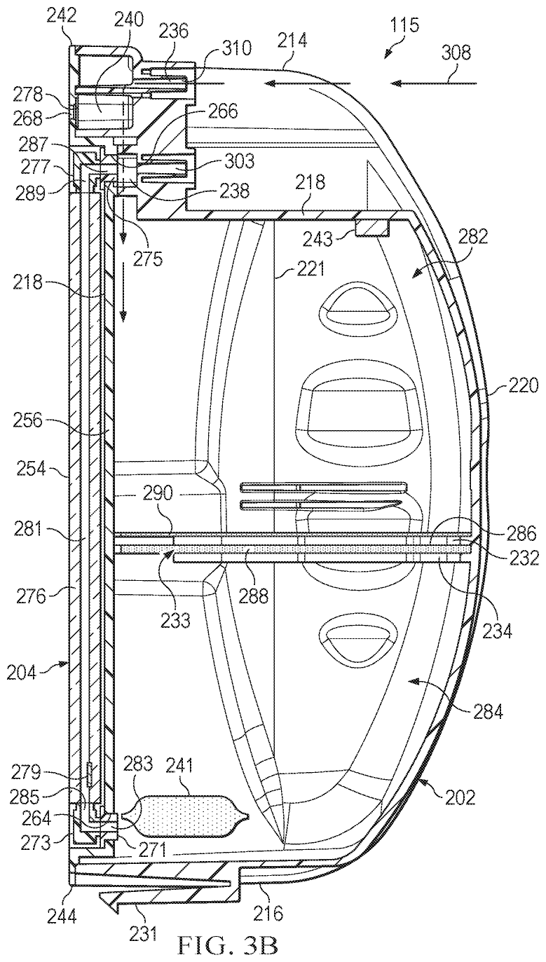

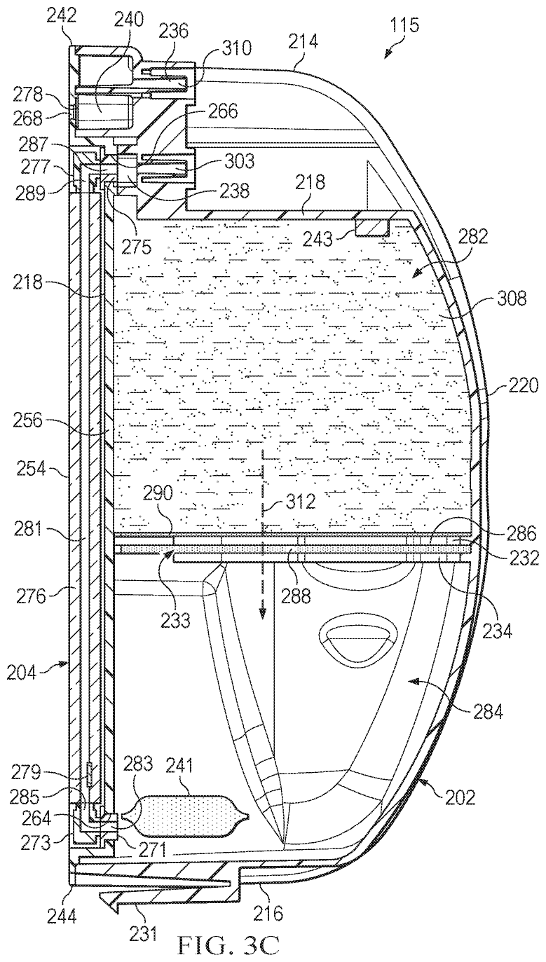

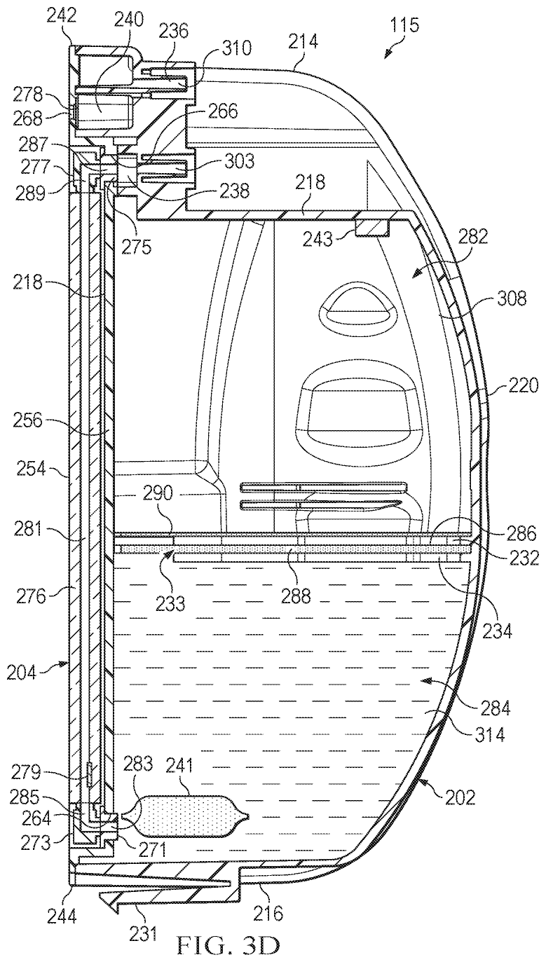

FIG. 3A is a sectional view of the recirculatory canister 115 of FIG. 2A taken along line 2E-2E of FIG. 2D and illustrating an instillation mode of operation. In FIG. 3A, fluid 302 is being transported from the second fluid chamber 284 through the first lumen 283 of the first piece 271, the second lumen 285 of the second piece 273, the lumen 281 of the conduit 276, the first lumen 287 of the first piece 275, the second lumen 289 of the second piece 277, and the fluid outlet 238. In some embodiments, a conduit, not pictured herein, may couple to the fluid outlet 238 and to the dressing 110 to couple the canister 115 to the dressing 110. In some embodiments, the path of the fluid flowing from the second fluid chamber 284 to the dressing 110 may be an instillation fluid pathway 303.

In some embodiments, the fluid 302 may be pre-filled in the second fluid chamber 284. For example, the fluid 302 may be added to the second fluid chamber 284 of the canister body 202 during assembly of the canister 115. In operation, the fluid 302 may be stored in the second fluid chamber 284 until it is desired to provide instillation therapy to the tissue site. When instillation therapy is desired, the controller 130 may be configured to operate the positive-pressure source 150 to draw the fluid 302 from the second fluid chamber 284 through instillation fluid pathway 303. In some embodiments, the controller 130 may operate the positive pressure source to move the fluid 302 through the instillation fluid pathway 303 to the dressing 110 until the second fluid chamber 284 is empty.

FIG. 3B is a cross-sectional view of the recirculatory canister 115 of FIG. 2A taken along line 2E-2E of FIG. 2D, illustrating a negative-pressure mode of operation. The canister 115 may be fluidly coupled between the negative-pressure source 105 and the dressing 110. The controller 130 may actuate the negative-pressure source 105, and fluid 308 from the dressing 110 may be drawn from the dressing 110 into the first fluid chamber 282. In some embodiments, negative-pressure source 105 may be configured to stop the negative-pressure mode of operation when the first sensor 243 detects a predetermined fill level of the first fluid chamber 282.

There may a conduit, not pictured herein, that may couple the dressing 110 to the fluid inlet 236. The fluid 308 may be drawn from the dressing 110, through the conduit, and into the first fluid chamber 282. The path that the fluid 308 takes from the dressing 110 to the first fluid chamber 282 may be a negative pressure pathway 310. The negative pressure pathway 310 may be offset from the instillation fluid pathway 303. For example, the negative pressure pathway 310 may direct the fluid 308 from the fluid inlet 236 towards the first side 222 of the canister body 202 to reach the first fluid chamber 282. By isolating the negative pressure pathway 310 from the instillation fluid pathway 303 the fluid 308 may not contaminate or contact the fluid 302.

FIG. 3C is a cross-sectional view of the recirculatory canister of FIG. 2A taken along line 2E-2E of FIG. 2D, illustrating fluids filtering from a first fluid chamber 282 of the canister 115 to a second fluid chamber 284 of the canister 115. During the negative-pressure mode of operation, the first fluid chamber 282 may fill with the fluid 308. The filter 206 may be configured to filter the fluid 308 from the first fluid chamber 282 to the second fluid chamber 284 as shown by arrow 312. The fluid 308 may flow through the filter 206 naturally with the force of gravity. In some embodiments, the fluid 308 may flow through the filter 206 due to a pressure differential between the first fluid chamber 282 and the second fluid chamber 284. More specifically, the fluid 308 begins in the first fluid chamber 282 and then may pass through the secondary filter 290, the filter carrier 286, and the primary filter 288 to reach the second fluid chamber 284.

The fluid 308 in the first fluid chamber 282 may be wound exudate from the tissue site being treated by the therapy system 100. As the fluid 308 passes through each element of the filter 206, the fluid 308 may be purified and cleansed such that contaminants such as bacteria, red blood cells, and/or viruses are removed by either the primary filter 288 or the secondary filter 290 and the filtered or purified fluid that enters the second fluid chamber 284 is of a quality that can either be disposed down a drain or may be instilled back into the tissue site being treated by the therapy system 100. In some embodiments, the filter 206 may convert the fluid 308 into saline or the fluid 302 may be converted into saline with the fluid altering device 241 of the second fluid chamber 284. In other embodiments, the filter 206 may be configured to allow certain elements of the fluid 308 that may be beneficial to the tissue site to pass through to the second fluid chamber 284 while capturing harmful or non-beneficial elements in at least one component of the filter 206. As discussed above, the filter 206 may filter fluid such that water (H₂O) molecules may pass through the filter 206 while larger bacterial molecules are captured by the filter 206. In some embodiments, the filter 206 may be sized to capture bacteria and red blood cells inhibiting passage of bacteria and red blood cells into the second fluid chamber 284. Additionally, one or more of the filter carrier 286, the primary filter 288, or the secondary filter 290 of the filter 206 may be positively or negatively charged to capture bacteria while allowing constituent parts of plasma to pass through the filter 206. In some embodiments, the filter 206 may also be configured to capture viruses to inhibit or prevent viruses from passing through the filter 206 into the second fluid chamber 284.

FIG. 3D is a cross-sectional view of the recirculatory canister of FIG. 2A taken along line 2E-2E of FIG. 2D, illustrating the second fluid chamber 284 of the canister 115 housing fluids. The second fluid chamber 284 may house fluid 314 that has traveled from the first fluid chamber 282 and through the filter 206 to reach the second fluid chamber 284. In some embodiments, the fluid 314 may be the same as or similar to the fluid 302 that was instilled into the tissue site from the second fluid chamber 284 as discussed with reference to FIG. 3A. In some embodiments, once the fluid 308 has all been filtered through the filter 206 to become the fluid 314 stored in the second fluid chamber 284, the therapy system 100 may be ready to instill the fluid 314 from the second fluid chamber 284 to the tissue site. The process described with reference to FIGS. 3A-3D may be repeated for as many cycles as necessary to treat the tissue site. In some embodiments, the process described with reference to FIGS. 3A-3D may be repeated provided the filter 206 is considered good quality. The filter may be considered good quality if the fluid passing through to the second fluid chamber 284 is of a desired quality. For example, if the filter 206 allows bacteria or other contaminants to pass into the second fluid chamber 284, the filter 206 may not be considered good quality. Additionally, if the filter 206 takes longer than a predetermined time to filter the fluid 308 from the first fluid chamber 282 to the second fluid chamber 284, the filter 206 may not be considered good quality. More specifically, if a predetermined quantity of the fluid 308 does not flow through the filter 206 in a predetermined amount of time, the filter 206 may be at the end of its lifespan and the filter 206 may need to be replaced.

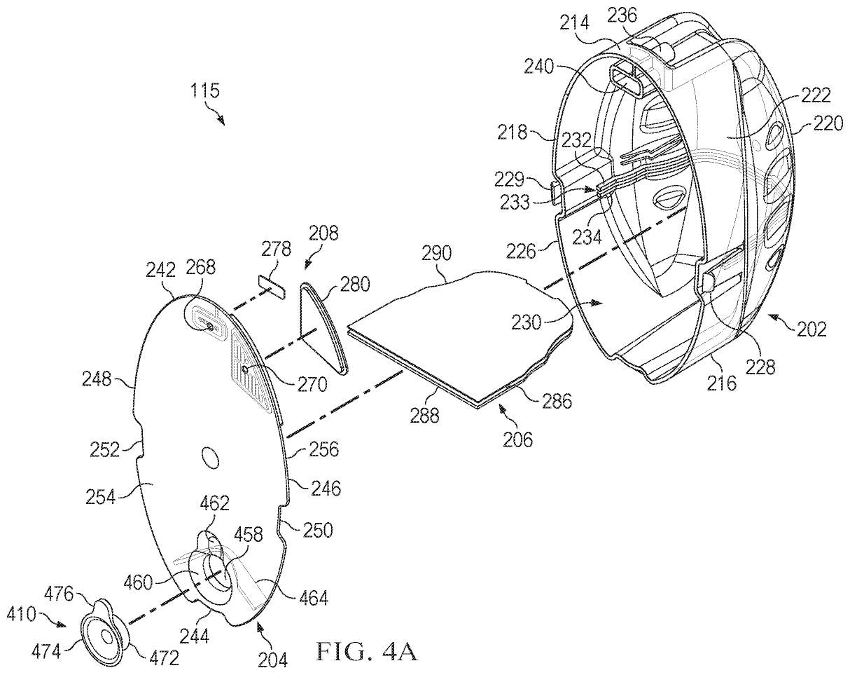

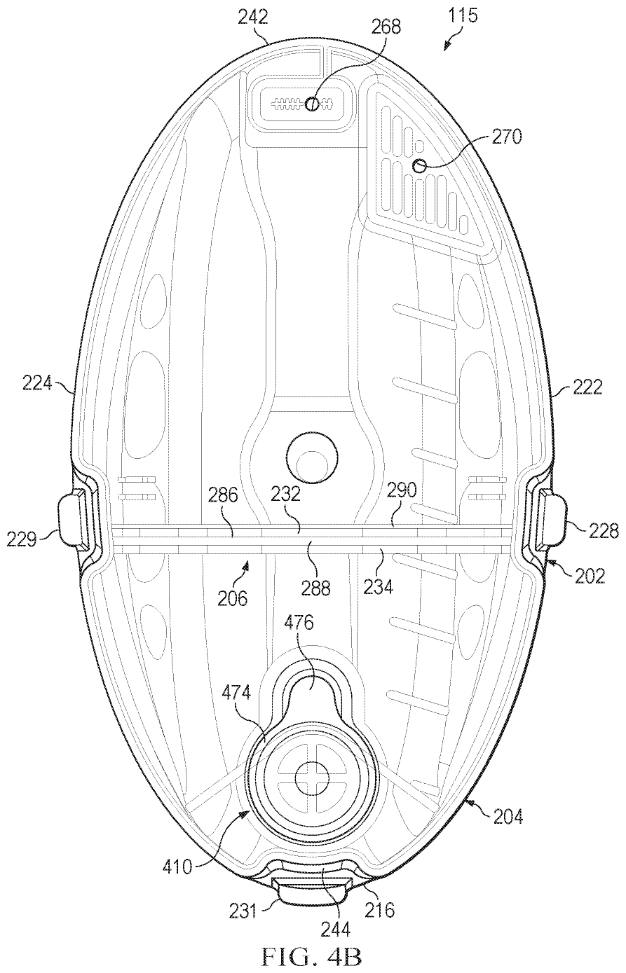

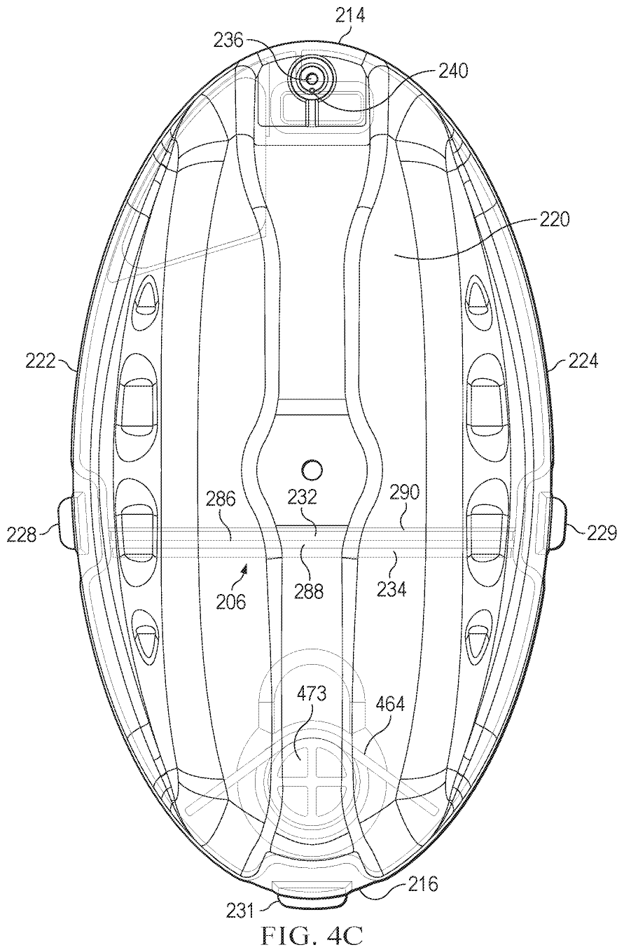

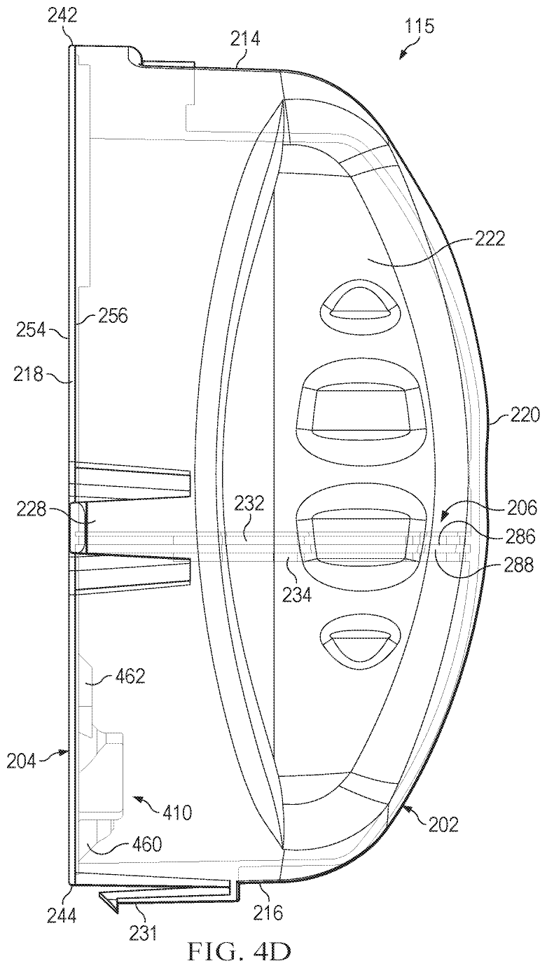

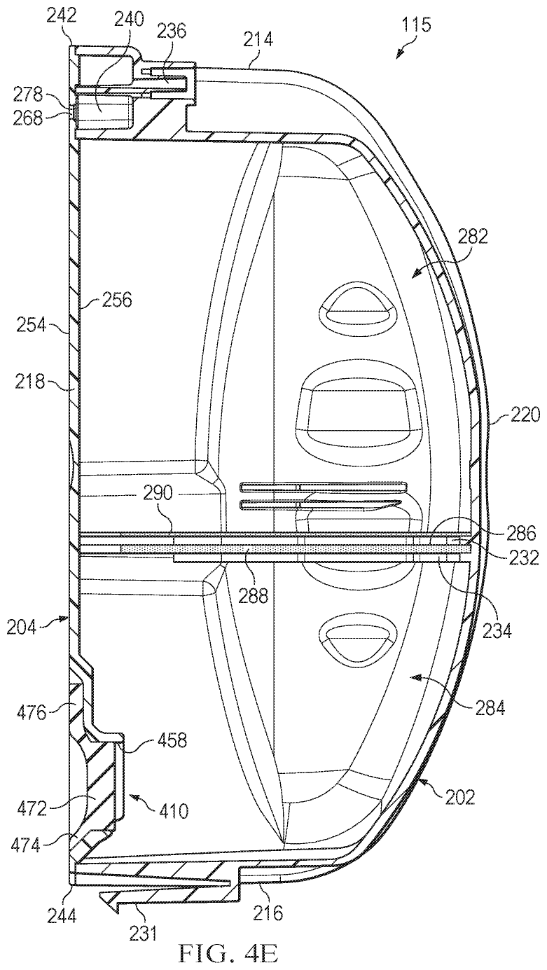

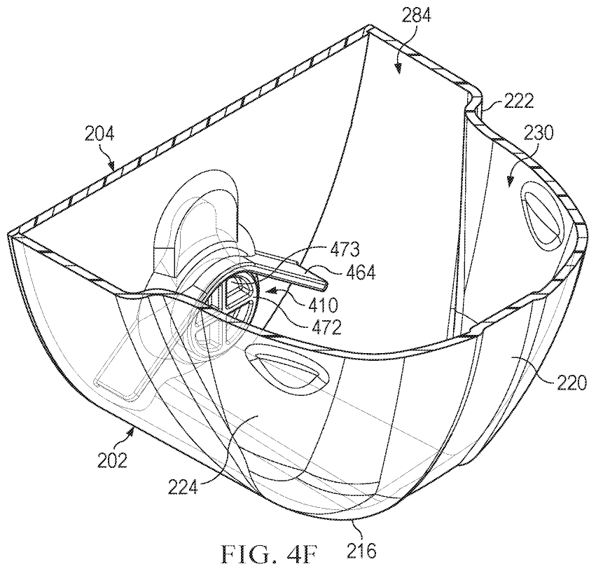

FIGS. 4A-4F illustrate various views of an exemplary embodiment of the canister 115 of the therapy system 100 of FIG. 1. In some embodiments, the canister 115 may be reusable. FIG. 4A is an exploded view of the canister 115 that may be associated with some embodiments of FIG. 1. FIG. 4B is a front view of the canister 115 of FIG. 4A. FIG. 4C is a back view of the canister 115 of FIG. 4A. FIG. 4D is a side view of the canister 115 of FIG. 4A. FIG. 4E is a cross-sectional view of the canister 115 of FIG. 4A taken along line 4E-4E of FIG. 4C. FIG. 4F is a cutaway view of a second fluid chamber of the reusable canister of FIG. 4A, illustrating additional details that may be associated with some example embodiments.

The canister 115 may include the canister body 202, the canister plate 204, the filter 206, the one or more negative pressure filters 208, and a plug 410. Although not pictured herein, the canister 115 may additionally include one or more of the fluid altering device 241, the first sterilization source 294, the second sterilization source 296, or the first sensor. In some embodiments, the fluid outlet 238 may be removed from the canister body 202. For example, the canister 115 may be a reusable canister that may receive fluids from a tissue site, be drained, and then be used again with therapy to receive more fluids form the tissue site. In these embodiments, instillation therapy may not be performed, and the canister body 202 may be formed without the fluid outlet 238.

In embodiments where the canister 115 may not be used to provide instillation therapy, the instillation system 210 may be removed. The canister plate 204 may include a port or an opening 458. The opening 458 may be disposed centrally between the first side 246 and the second side 248 and proximate to the second end 244. The opening 458 may extend through the canister plate 204, permitting fluid communication across the canister plate 204. In some embodiments, an annular wall 459 may be coupled to the interior surface 256. The annular wall 459 may surround the opening 458 and depend into the interior 230 of the canister 115. In some embodiments, a bevel, a chamfer, or a fillet may be disposed at the union of the annular wall 459 with the opening 458, forming an indentation 460 that may surround the opening 458. In some embodiments, a recess 462 or cavity may be formed in the canister plate 204 proximate to the opening 458. The recess 462 may depend into the canister plate 204. In some embodiments, the recess 462 may not permit fluid communication across the canister plate 204 with the interior 230 of the canister 115. In some embodiments, a portion of the recess 462 may be coupled to the indentation 460. In some embodiments, the opening 458 may be configured to receive the plug 410.

The canister plate 204 may also include a structure, such as a baffle 464. The baffle 464 may be coupled to the interior surface 256 and depend into the interior 230 of the canister 115. In some embodiments, the baffle 464 may be disposed on the interior surface 256 proximate to the opening 458 and the annular wall 459. The baffle 464 may have a height greater than the annular wall 459. The baffle 464 may have an inverted V-shape having a central potion aligned with a center of the opening 458. The baffle 464 may have two lateral portions, a first extending toward and terminating proximate to the first side 246 and a second extending toward and terminating proximate to the second side 248. In some embodiments, distal ends of the lateral portions may be disposed between the central portion of the baffle 464 and the second end 244 of the canister plate 204.

The plug 410 may be configured to seat within the opening 458 and prevent fluid communication through the opening 458. In some embodiments, the plug 410 may include a stopper 472. In some embodiments, the stopper 472 may be a cylindrical body having a diameter substantially equal to a diameter of the opening 458. The stopper 472 may have a height substantially equal to a height of the annular wall 459, so that, if the stopper 472 is seated within the opening 458, an end of the stopper 472 may be flush with an end of the annular wall 459. In some embodiments, the stopper 472 may include one or more indentations 473 depending into the stopper 472. When the canister 115 is assembled, the indentations 473 may be exposed to the interior 230 and may be in contact with fluid stored within the canister 115. The plug 410 may additionally include a sealing ring 474 coupled to the stopper 472. The sealing ring 474 may be coupled to an end of the stopper 472 opposite the indentations 473. The plug 410 may also include a gripping portion 476 coupled to the sealing ring 474. In operation, the plug 410 may be inserted into the opening 458. The stopper 472 may couple to the canister plate 204 in an interference fit, preventing fluid flow through the opening 458. The sealing ring 474 may be in contact with the indentation 460, providing a further seal between the canister plate 204 and the plug 410. The gripping portion 476 may fit within the recess 462 so that the plug 410 may be flush with the exterior surface 254 of the canister plate 204. The plug 410 may provide a fluid seal at the opening 458 of the canister plate 204 preventing fluid communication across the canister plate 204 with the interior 230 through the opening 458. In some embodiments, the plug 410 may be removable, in other embodiments, the plug 410 may be permanently attached to the canister plate 204. The plug 410 may be formed from any of the materials described above with reference to the canister body 202 and the canister plate 204.

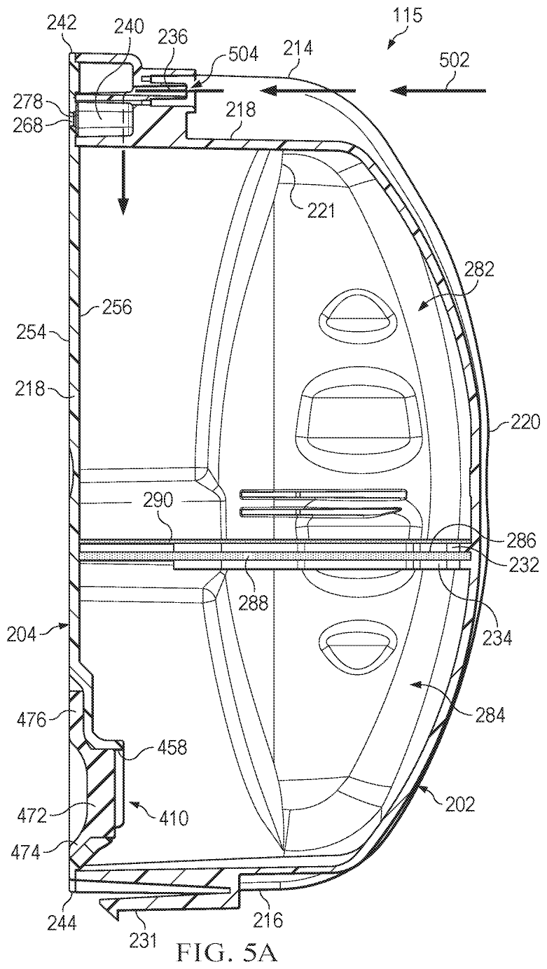

FIG. 5A is a cross-sectional view of the canister 115 of FIG. 4A taken along line 4E-4E of FIG. 4C, illustrating a negative-pressure mode of operation. The negative-pressure mode of operation may be substantially similar to the negative-pressure mode of operation described above with reference to FIG. 3B. For example, there may a conduit, not pictured herein, that may couple the dressing 110 to the fluid inlet 236. Fluid 502 may be drawn from the dressing 110, through the conduit, and into the first fluid chamber 282. The path that the fluid 502 takes from the dressing 110 to the first fluid chamber 282 may be a negative pressure pathway.

FIG. 5B is a cross-sectional view of the canister 115 of FIG. 4A taken along line 4E-4E of FIG. 4C, illustrating the negative-pressure mode of operation while the fluid 502 is filtering from the first fluid chamber 282 of the canister 115 to the second fluid chamber 284 of the canister 115. In FIG. 5B, the negative-pressure source 105 is operating and the first fluid chamber 282 is partially filled with the fluid 502. The second fluid chamber 284 is partially filled with fluid 508 that has been filtered through the filter 206. Arrow 510 may represent the process of the fluid 502 being filtered through the filter 206. The process of the fluid 502 filtering through the filter 206 may be substantially similar to the process described above with reference to FIG. 3C.

The fluid 502 may continue to filter through the filter 206 until the second fluid chamber 284 is substantially full of the fluid 508. In some embodiments, the first sensor 243 may be configured to determine when the second fluid chamber 284 is full. In other embodiments, the canister 115 may include an additional sensor that may be configured to determine when the second fluid chamber 284 is full.

FIG. 5C is a cross-sectional view of the canister 115 of FIG. 4A taken along line 4E-4E of FIG. 4C, illustrating fluids being removed from the second fluid chamber 284 in an emptying mode. The emptying mode may follow the negative-pressure mode of operation such that the fluid 502 is not flowing into the first fluid chamber 282 during the emptying mode. During the emptying mode, the plug 410 may be removed from the opening 458. For example, a user may separate the canister 115 from the therapy system 100, exposing the plug 410. The user may grip the gripping portion 476 and apply a force to the plug 410, unseating the sealing ring 474 and the stopper 472 from the indentation 460 and the opening 458, respectively. If the plug 410 is removed from the canister 115, the fluid 508 in the second fluid chamber 284 may flow through the opening 458 to exit the canister 115. Arrow 512 may represent the flow of the fluid 508 out of the second fluid chamber 284. The fluid 508 may be of a quality that it can be disposed of in a standard drain and does not need to be treated as medical waste. The purification of the fluid 502 into the fluid 508 by the filter 206 may allow a user to empty the canister 115 and continue to use the canister 115 and the therapy system 100 after the canister 115 has been emptied. In some embodiments, the canister 115 may allow a user to repeatedly operate multiple negative-pressure therapy cycles with the therapy system 100 in a home setting. For example, as the canister 115 is filled, the user may dispose of the fluid 508 from the second fluid chamber 284, reattach the canister 115 to the therapy system 100 and continue therapy.

The process described with reference to FIGS. 5A-5D may be repeated until therapy of the tissue site concludes. In some embodiments, the process may be repeated until the life cycle of the filter 206 is reached. For example, if a predetermined quantity of the fluid 502 does not flow through the filter 206 in a predetermined amount of time, the filter 206 may be at the end of its lifespan and the filter 206 may be replaced.

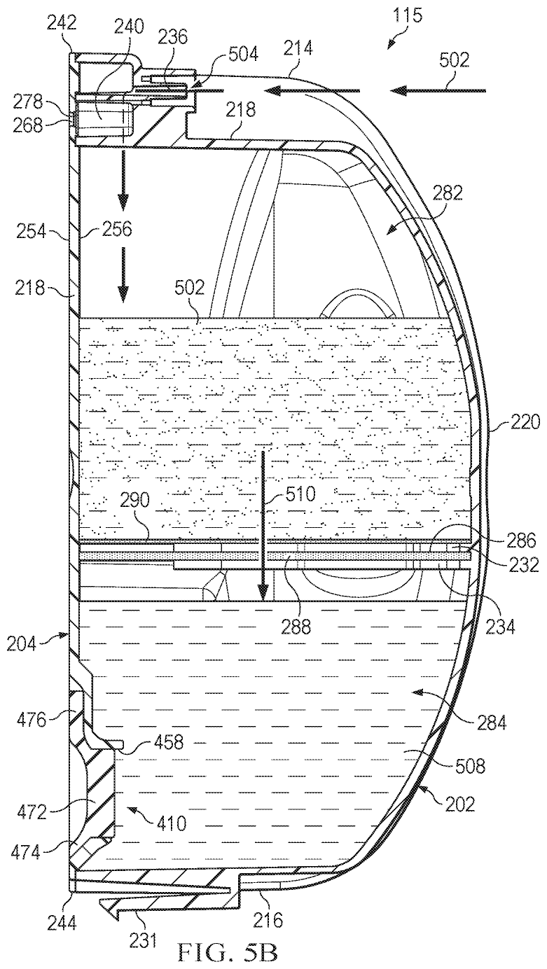

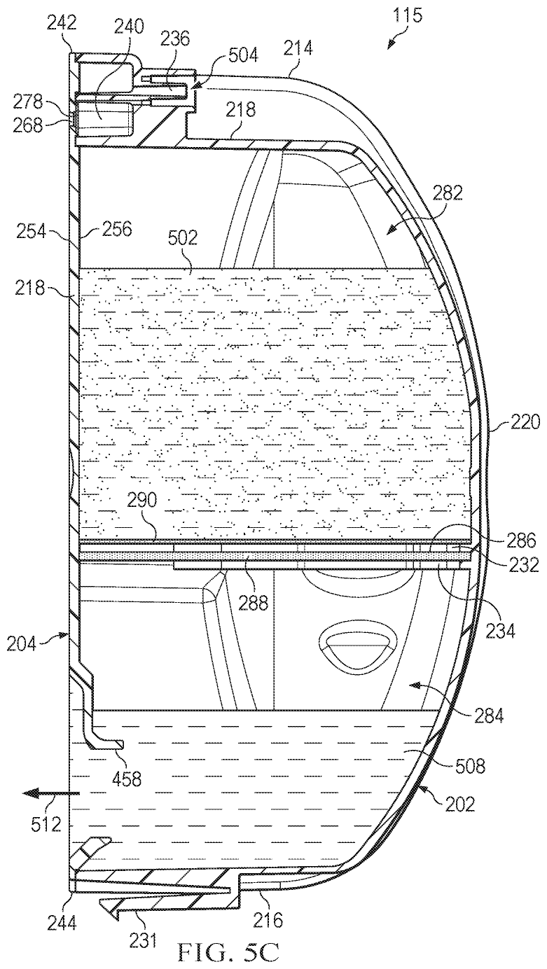

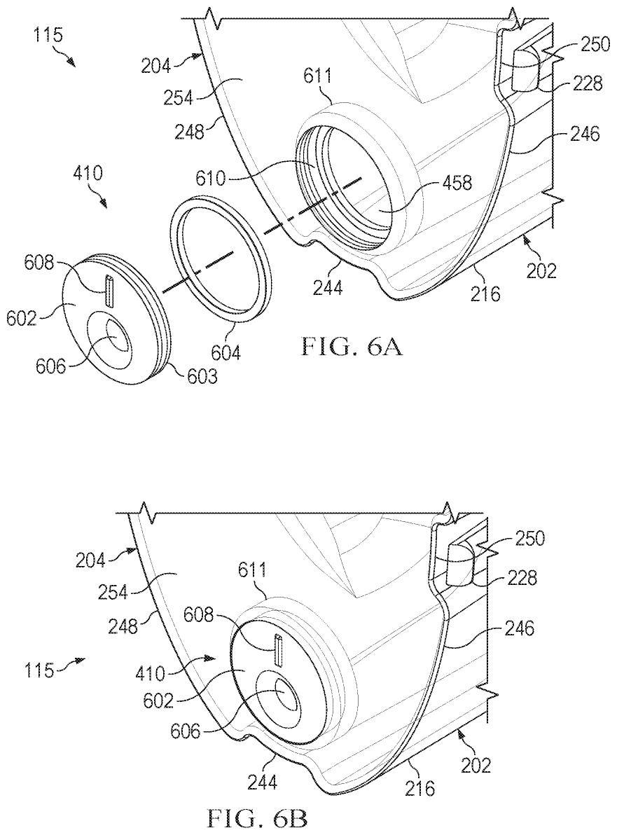

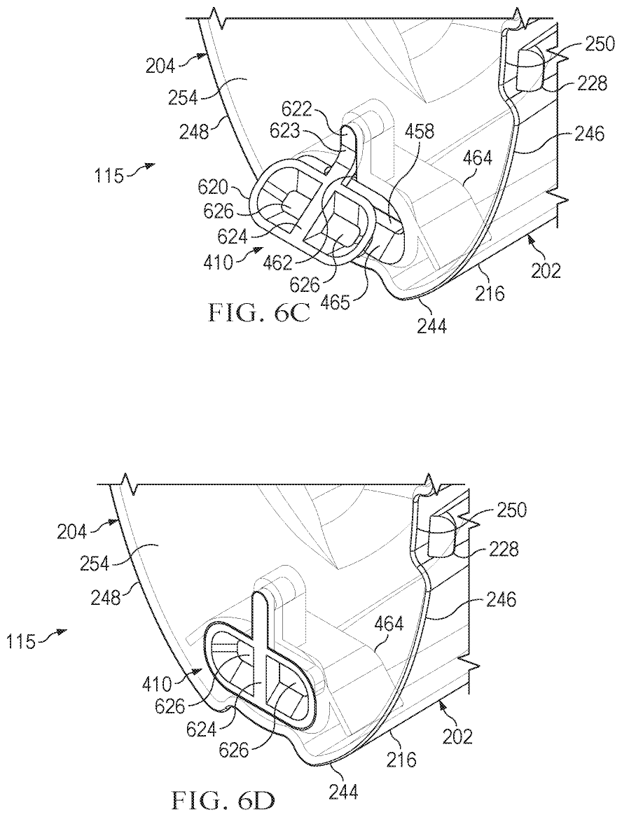

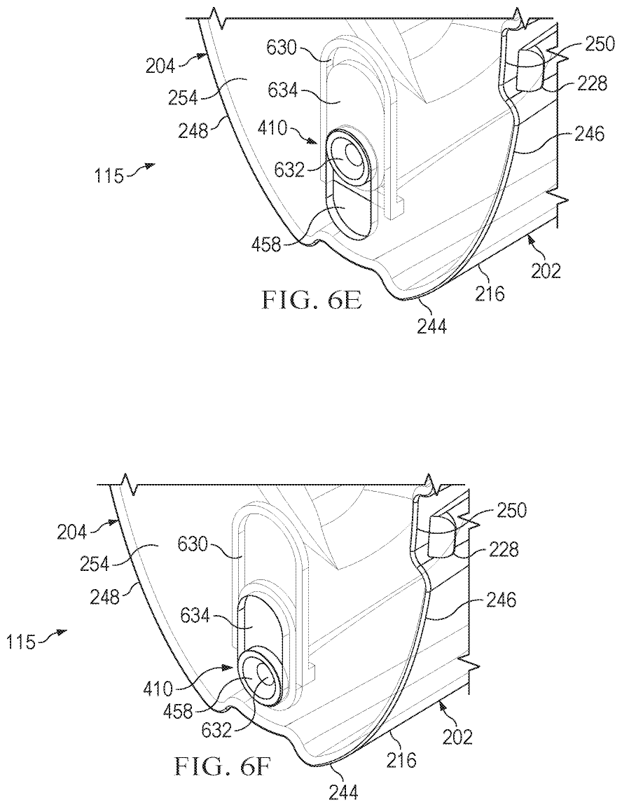

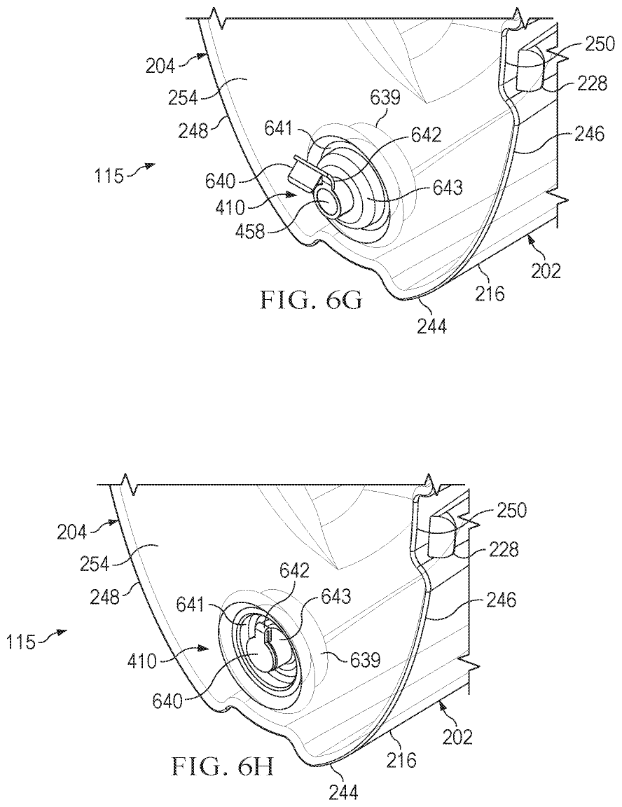

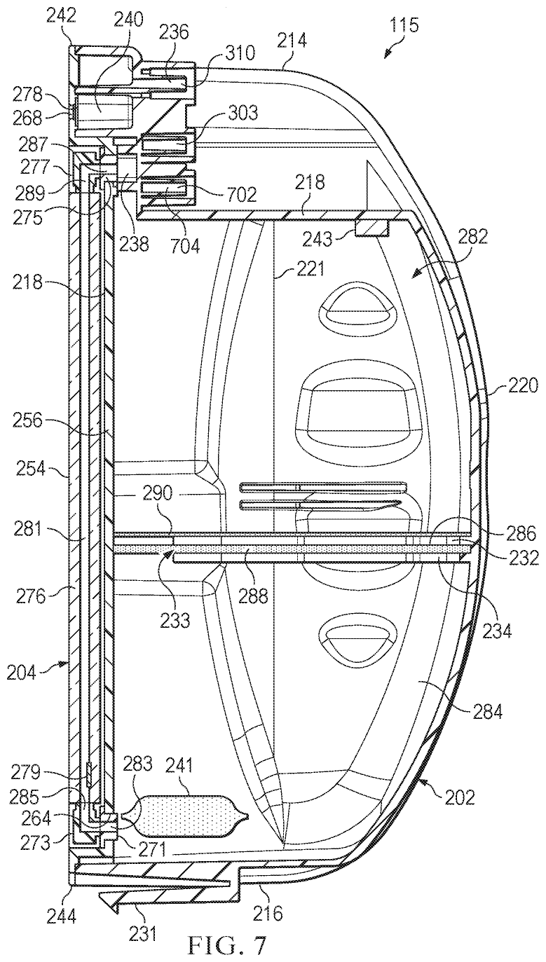

FIG. 6A is an assembly view of another embodiment of the plug 410 and a portion of the canister plate 204 illustrating additional details that may be associated with some embodiments. In some embodiments, the opening 458 may be a straight hole formed through the canister plate 204. The opening 458 may be bounded by a substantially cylindrical wall having a thread 610 formed thereon. In some embodiments, the opening 458 may have a seat 611 formed in the opening 458 proximate to the interior surface 256. The seat 611 may provide a shelf having a surface facing away from the interior 230.

In some embodiments, the plug 410 is configured to be coupled to the canister plate 204 at the opening 458 through a pair of mating threads. The plug 410 may include a fastener 602 and a seal 604. In some embodiments, the fastener 602 may be disc-shaped body having a side wall. In some embodiments, the side wall may include a thread 603. The thread 603 may be configured to mate with the thread 610. In some embodiments, the fastener 602 may include an indentation 606 and a projection 608. For example, the indentation 606 may be disposed in a surface of the fastener 602 configured to face away from the interior 230. The indentation 606 may depend into the surface of the fastener 602. In some embodiments, the indentation 606 may be disposed proximate to an edge of the surface of the fastener 602. In other embodiments, the indentation 606 may be disposed proximate to a center of the surface of the fastener 602. The projection 608 may be disposed on the surface of the fastener 602 configured to face away from the interior 230. The projection 608 may be aligned with the indentation 606. For example, the projection 608 may have an axis that is aligned with a center of the indentation 606. In some embodiments, the indentation 606 and the projection 608 may provide texture to the surface of the fastener 602, permitting a user to apply sufficient force to the fastener 602 so that the fastener 602 may be secured to and removed from the canister plate 204.

The seal 604 may be a ring configured to fit within the opening 458 of the canister plate 204. The seal 604 may be configured to be disposed on the seat 611 within the opening 458. The fastener 602 may be secured to the canister plate 204 through the thread 603 and the thread 610, compressing the seal between the fastener 602 and the seat 611.

FIG. 6B is a perspective view of the plug 410 and a portion of the canister plate 204 of FIG. 6A, illustrating additional details that may be associated with some embodiments. As illustrated in FIG. 6B, the fastener 602 may be turned in a clockwise or a counterclockwise motion when the fastener 602 is adjacent to the opening 458. The thread 603 may engage the thread 610 of the opening 458 to secure the plug 410 to the canister plate 204. In some embodiments, the surface of the fastener 602 having the indentation 606 and the projection 608 may be flush with the exterior surface 254 of the canister plate 204.

FIG. 6C is an assembly view of another embodiment of the plug 410 and a portion of the canister plate 204 illustrating additional details that may be associated with some embodiments. As illustrated in FIG. 6C, the opening 458 may have a generally rectangular shape with semi-circular ends. Generally, linear sides of the opening 458 may be oriented to be proximate to the second end 244. One each of the semi-circular ends may be proximate to a respective first side 246 and a second side 248. In some embodiments, the recess 462 may extend from the opening 458 toward the first end 242 of the canister plate 204.

In some embodiments, the plug 410 may have a first section 620 and a second section 622. The first section 620 may be shaped to mate with the opening 458 so that the first section 620 may be inserted into the opening 458. In some embodiments, the first section 620 may be configured to substantially fill the opening 458, sealing the opening 458. In some embodiments, the first section 620 may include a central portion 624 and one or more gripping portions 626 surrounding the central portion 624. The second section 622 may extend from the first section 620. For example the second section may have a first end configured to be coupled to a linear side of the first section 620. The second section 622 may extend away from the first section so that a second end of the second section 622 may be separated from the first section 620. In some embodiments, the second end of the second section 622 may be coupled to the canister plate 204 proximate to the opening 458. For example, the second end of the second section 622 may be disposed in the recess 462 and coupled to the canister plate 204. In some embodiments, the second section 622 may include a hinge 623. The hinge 623 may be disposed between the first end and the second end of the second section 622. In some embodiments, the hinge 623 may be configured to permit the first section 620 to swing away from the canister plate 204 along at least one axis of rotation.

FIG. 6D is a perspective view of the plug 410 and a portion of the canister plate 204 of FIG. 6C, illustrating additional details that may be associated with some embodiments. As illustrated in FIG. 6D, the second section 622 may couple the plug 410 to the canister plate 204 such that when the first section 620 is removed from the opening 458, the plug 410 may remain connected to the canister plate 204. To remove the plug 410 from the opening 458, a user may grip the central portion 624 by inserting one or more fingers into the one or more gripping portions 626 and may pull the first section 620 from the opening 458. The second section 622 may remain in contact with the canister plate 204 such that the plug 410 is not lost or discarded while the fluid is being removed from the canister 115. In some embodiments, the baffle 464 may be shaped accommodate the opening 458 of FIG. 6C and FIG. 6D.

FIG. 6E is an assembly view of another embodiment of the plug 410 and a portion of the canister plate 204 illustrating additional details that may be associated with some embodiments. The plug 410 and the opening 458 may comprise a sliding door type mechanism. As illustrated in FIG. 6E, the plug 410 may be in an open position. The opening 458 may generally be rectangular having semi-circular ends. The linear portions of the opening 458 may be oriented to be proximate to the first side 246 and the second side 248, respectively. In some embodiments, the semi-circular portions may be oriented to be proximate the first end 242 and the second end 244 of the canister plate 204. The semi-circular portions of the opening 458 may have a diameter substantially equal to a diameter of the plug 410, permitting the plug 410 to fit within the opening 458. A plug section 630 may be built into the canister plate 204 such that at least a portion of the plug 410 may be within the plug section 630 of the canister plate 204 in both the closed position and the open position of the plug. The plug section 630 may extend from the opening 458 towards the first end 242 of the canister plate 204. The plug section 630 may be a hollow portion of the canister plate 204 between the exterior surface 254 and the interior surface 256 of the canister plate 204.

The plug 410 may include a contact portion 632 and a sliding portion 634. The contact portion 632 may extend past the exterior surface 254 such that a user may engage with the contact portion 632. The sliding portion 634 may be received by the plug section 630 of the canister plate 204. The sliding portion 634 may partially extend into the plug section 630 when the plug 410 is configured to close the opening from an ambient environment. The sliding portion 634 may be fully inserted into the plug section 630 when the opening 458 is exposed to allow fluid to be removed from the canister 115. By sliding within the plug section 630 to close and expose the opening 458, the plug 410 may be configured to remain in contact with the canister plate 204 such that the plug 410 is not lost or discarded while the fluid is being removed from the canister 115.

FIG. 6G is an assembly view of another embodiment of the plug 410 and a portion of the canister plate 204 illustrating additional details that may be associated with some embodiments. In some embodiments, the opening 458 may be a straight hole formed through the canister plate 204. The opening 458 may be bounded by a substantially circular wall configured to house the plug 410. In some embodiments, the opening 458 may include a seat 639 formed in the opening 458 proximate to the interior surface 256 of the canister plate 204.

In some embodiments, the plug 410 may include an outer wall 641 that may engage the seat 639 of the opening 458. The plug 410 may include a cap 640 that may be coupled to a body 643 of the plug 410 with a connector 642. In some embodiments, the connector 642 may be a hinge that allows the cap 640 to be opened while remaining connected to the body 643 of the plug 410.

FIG. 6H is a perspective view of the plug 410 and a portion of the canister plate 204 of FIG. 6A, illustrating additional details that may be associated with some embodiments. As illustrated in FIG. 6H, the cap 640 may be removed from the body 643 of the plug 410. In some embodiments, the body 643 of the plug 410 may be pulled towards the exterior surface 254 away from the interior surface 256 of the canister plate 204 for easier access to the cap 640.

In any of the embodiments of FIGS. 6A-6H, the plug 410 may utilize a negative pressure in the canister 115 while the negative-pressure source 105 is operating to fluidly seal the canister 115. Additionally, the plug 410 may act as a spout to improve control when emptying the canister 115. In some embodiments, the baffle 464 may also help to control the fluid as is it exiting the canister 115 through the opening 458. In other embodiments, the plug 410 and the opening 458 may different sizes, shapes, and configurations but may all maintain a fluid seal such that fluid cannot escape from the canister 115 when the plug 410 is sealing the opening 458.

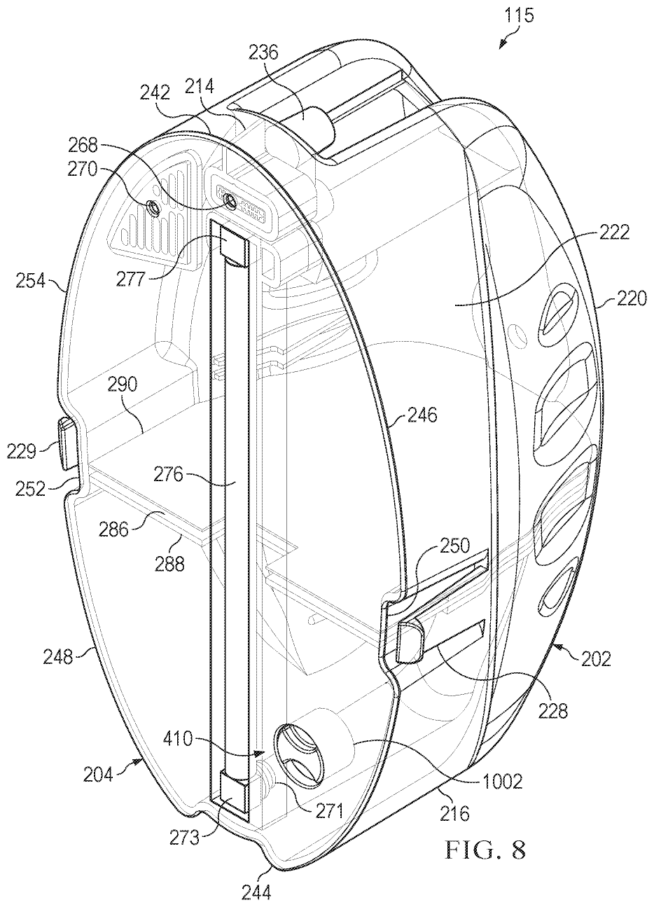

Referring to FIG. 7, another embodiment of an exemplary embodiment of the canister 115 of the therapy system 100 of FIG. 1 is shown. The canister 115 may be a recirculatory canister similar to the canister 115 shown in FIGS. 2A-2E and 3A-3D. The canister 115 may be substantially similar to the canister of FIGS. 2A-2E and 3A-3D but may include a fill inlet 702 that may be fluidly coupled to the second fluid chamber 284 through a fill pathway 704. that may be isolated from both the instillation fluid pathway 303 and the negative pressure pathway 310. The fill pathway 704 may extend from the second fluid chamber 284, through the canister body 202 to the fill inlet 702 to connect to a conduit, not pictured herein, that may be coupled to an external fluid source. In some embodiments, the fill pathway 704 may include components of the instillation system 210. For example, the fill pathway 704 may extend from the second fluid chamber 284 through the first lumen 283 of the first piece 271, the second lumen 285 of the second piece 273, the lumen 281 of the conduit 276, the first lumen 287 of the first piece 275, the second lumen 289 of the second piece 277, the fluid outlet 238, and the fill inlet 702 to reach the conduit coupling the fill inlet to the external fluid source. The fill pathway 704 may be isolated from the first fluid chamber 282 such that fluid from the external fluid source is inserted into the second fluid chamber 284 without contacting the filter 206 or the first fluid chamber 282.

Referring to FIG. 8, another embodiment of an exemplary embodiment of the canister 115 of the therapy system 100 of FIG. 1 is shown. The canister 115 may be a combination recirculatory and reusable canister. In some embodiments, the canister 115 may be substantially similar to the canister of FIGS. 2A-2E and 3A-3D but may include the plug 410 of any of the embodiments of the canister 115 of FIGS. 4A-4F, 5A-C, and 6A-6H. The canister 115 may be configured to instill fluid from the second fluid chamber 284 to the dressing 110 or to empty fluid from the second fluid chamber 284 through the opening 458 the canister plate 204. In some embodiments, the opening 458 may be configured to couple with the plug 410 and may include an extension or a spout 1002 that extends from the interior surface 256 of the canister plate 204 away from the exterior surface 254 of the canister plate 204. The spout 1002 may be configured to facilitate the removal of fluid from the second fluid chamber 284.

In some embodiments, the canister 115 may be capable of functioning as described above with reference to FIGS. 3A-3D. Additionally, during the operation of the therapy system 100, the plug 410 may be removed from the canister plate 204 to remove fluid from the second fluid chamber 284. For example, if the fluid in the second fluid chamber 284 is of a quality that should not be instilled back to the tissue site, the fluid may be removed through the spout 1002 of the canister 115. Additionally or alternatively, if the tissue site is done being treated with the therapy system 100, the fluid may be removed from the second fluid chamber 284 to dispose of the fluid.

In some embodiments, the opening 458 may be configured to transport fluid from an external fluid source into the second fluid chamber 284. More specifically, the plug 410 may be removed from the opening 458 to expose the interior 230 of the canister body 202 such that fluid from an external fluid source may be poured into the canister 115. After the fluid has been disposed within the second fluid chamber 284, the plug 410 may be reinserted into the opening 458 to seal the canister 115. The canister 115 may then be used with the therapy system 100 to treat a tissue site as described above with reference to FIGS. 3A-3D.

Also described herein is a method of treating a tissue site. The method can include disposing the dressing 110 at a tissue site, fluidly coupling the negative-pressure source 105 to the dressing 110, and fluidly coupling the canister 115 between the negative-pressure source 105 and the dressing 110. The canister 115 can include the first fluid chamber 282, the second fluid chamber 284, and the filter 206. The first fluid chamber 282 can be configured to collect fluids from the tissue site. The filter 206 can be disposed between the first fluid chamber 282 and the second fluid chamber 284. The filter 206 can be configured to filter the fluids from the tissue site as the fluids move through the filter 206 from the first fluid chamber 282 to the second fluid chamber 284. The method can further include operating the negative-pressure source 105 to generate a negative pressure at the dressing 110, drawing fluids from the tissue site into the first fluid chamber 282 of the canister 115 in response to the negative pressure, and filtering, with the filter 206, the fluids from the tissue site as the fluids move from the first fluid chamber 282 to the second fluid chamber 284.

In some embodiments, the method can further include instilling the filtered fluids of the second fluid chamber 284 to the tissue site. In some embodiments, the method can further include disposing of the filtered fluids of the second fluid chamber 284. In some example embodiments, disposing of the filtered fluids of the second fluid chamber 284 can include removing the plug 410 from an emptying port such as the opening 458 of the second fluid chamber 284 and drawing the filtered fluids from the second fluid chamber 284 through the emptying port.

In some example embodiments, the method can further include purifying the fluids in the first fluid chamber 282 with a UV-C source such as the first sterilization source 294 and the second sterilization source 296. In some example embodiments, the method can further include treating the filtered fluids of the second fluid chamber 284 with a UV-C source such as the first sterilization source 294 and the second sterilization source 296. In some example embodiments, the second fluid chamber 284 can be configured to receive and house instillation fluid.

In some example embodiments, the method can further include filling the second fluid chamber 284 with instillation fluid. In some example embodiments, the method can further include fluidly coupling the second fluid chamber 284 to the dressing 110 and instilling the instillation fluid from the second fluid chamber 284 to the dressing 110. In some example embodiments, filling the second fluid chamber 284 with the instillation fluid can include adding the instillation fluid from an external fluid source through a port such as the opening 458 of the second fluid chamber 284. In some example embodiments, filling the second fluid chamber 284 with instillation fluid includes coupling a fluid pathway such as the instillation fluid pathway 303 of the canister 115 to an external fluid source and operating a pump such as the positive-pressure source 150 to draw fluid from the external fluid source to the second fluid chamber 284.

The systems, apparatuses, and methods described herein may provide significant advantages. For example, the embodiments of the canister 115 described herein result in fewer canister changes, reduced size for the therapy system 100, and simpler systems for users and health care practitioners to manage. Additionally, the embodiments of the canister 115 described herein result in reduced waste because the filter 206 may cleanse and purify fluids from the tissue site such that the fluids can be disposed of down a drain and do not have to be treated as medical waste.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the canister 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A canister for use in a negative pressure wound therapy system, the canister comprising:
a first fluid chamber configured to receive fluids from a tissue site;
a second fluid chamber; and
a filter disposed between the first fluid chamber and the second fluid chamber, the filter configured to filter the fluids from the tissue site as the fluids move from the first fluid chamber to the second fluid chamber.

2. The canister of claim 1, wherein the filter comprises:
a filter carrier configured to be coupled to the canister between the first fluid chamber and the second fluid chamber;
a primary filter coupled to the filter carrier and disposed proximate to the second fluid chamber; and
a secondary filter coupled to the filter carrier and disposed proximate to the first fluid chamber.

3. The canister of claim 1, further comprising an instillation fluid pathway configured to fluidly couple the second fluid chamber to the tissue site.

4. The canister of claim 3, wherein the instillation fluid pathway is disposed along an exterior of the canister.

5. The canister of claim 3, wherein the instillation fluid pathway is isolated from the first fluid chamber.

6. The canister of claim 3, further comprising a negative pressure pathway configured to fluidly couple the first fluid chamber to the tissue site.

7. The canister of claim 6, wherein the negative pressure pathway is isolated from the instillation fluid pathway.

8. The canister of claim 6, further comprising a fill pathway configured to fluidly couple the second fluid chamber to an external fluid source, the fill pathway being isolated from the negative pressure pathway and the instillation fluid pathway.

9. (canceled)

10. The canister of claim 3, further comprising a sensor disposed in the instillation fluid pathway, the sensor being configured to generate a signal representative of a fill status of the second fluid chamber.

11. The canister of claim 1, further comprising a first sterilization source configured to sterilize the first fluid chamber and a second sterilization source configured to sterilize the second fluid chamber.

12. (canceled)

13. The canister of claim 1, further comprising a fluid altering device disposed within the second fluid chamber, the fluid altering device configured to release chemicals into fluids disposed within the second fluid chamber to at least one of sterilize the fluids within the second fluid chamber or change properties of the fluids within the second fluid chamber.

14. (canceled)

15. (canceled)

16. The canister of claim 1, further comprising a negative pressure filter disposed within the first fluid chamber and configured to prevent liquids from the tissue site from contacting a negative pressure source.

17. The canister of claim 1, wherein the second fluid chamber comprises a port disposed at an end of the second fluid chamber opposite the first fluid chamber.

18. (canceled)

19. The canister of claim 17, further comprising a plug configured to removably couple to the port.

20. The canister of claim 17, wherein the port includes a spout.

21. The canister of claim 1, further comprising a sensor disposed in the first fluid chamber, the sensor being configured to generate a signal representative of a fill status of the first fluid chamber.

22. A system for treating a tissue site, the system comprising:
a dressing configured to be disposed at the tissue site;
a negative-pressure source configured to be fluidly coupled to the dressing and further configured to generate a negative pressure at the tissue site; and
a canister configured to be fluidly coupled between the dressing and the negative-pressure source, the canister comprising:
a first fluid chamber configured to receive fluids from the tissue site;
a second fluid chamber configured to store fluids; and
a filter disposed between the first fluid chamber and the second fluid chamber, the filter configured to filter the fluids from the tissue site as the fluids move through the filter from the first fluid chamber to the second fluid chamber.

23. The system of claim 22, further comprising an instillation fluid pathway configured to fluidly couple the second fluid chamber to the tissue site and a negative pressure pathway configured to fluidly couple the negative-pressure source to the dressing and the first fluid chamber of the canister, the negative pressure pathway being isolated from the instillation fluid pathway.

24. (canceled)

25. A method of treating a tissue site, the method comprising:
disposing a dressing at the tissue site;
fluidly coupling a negative-pressure source to the dressing;
fluidly coupling a canister between the negative-pressure source and the dressing, the canister comprising, a first fluid chamber configured to collect fluids from the tissue site;
a second fluid chamber; and
a filter disposed between the first fluid chamber and the second fluid chamber, the filter configured to filter the fluids from the tissue site as the fluids move through the filter from the first fluid chamber to the second fluid chamber;
operating the negative-pressure source to generate a negative pressure at the dressing;
drawing fluids from the tissue site into the first fluid chamber of the canister in
response to the negative pressure; and
filtering, with the filter, the fluids from the tissue site as the fluids move from the first fluid chamber to the second fluid chamber.

26. The method of claim 25, further comprising instilling the filtered fluids of the second fluid chamber to the tissue site.

27.-36. (canceled)
